(19) United States
(12) Patent Application Publication
(10) Pub. No.: US 2010/0184790 A1
(43) Pub. Date: Jul. 22, 2010

(54) PYRROLO[2,3-B]PYRIDINE COMPOUNDS, AZAINDOLE COMPOUNDS USED FOR SYNTHESIZING SAID PYRROLO[2,3-B]PYRIDINE COMPOUNDS, METHODS FOR THE PRODUCTION THEREOF, AND USES THEREOF

(75) Inventors: Laurent Meijer, Roscoff (FR); Benoit Joseph, Villeurbanne (FR); Francois Liger, Villeurbanne (FR); Bernard Marquet, Caluire (FR)

Correspondence Address: OLIFF & BERRIDGE, PLC, P.O. BOX 320850, ALEXANDRIA, VA, 22320-4850, US

(73) Assignees: CENTRE NATIONAL DE LA RECHERCHE SCIENTIFIQUE, Paris (FR); UNIVERSITE CLAUDE BERNARD LYON 1, Villeurbanne (FR); UNIVERSITE PIERRE ET MARIE CURIE (PARIS 6), Paris (FR)

(21) Appl. No.: 12/526,446

(22) PCT Filed: Feb. 14, 2008
(86) PCT No.: PCT/FR2008/000197

§ 371 Date: Mar. 8, 2010

(30) Foreign Application Priority Data
Feb. 16, 2007 (FR) 07 01138

(51) Int. Cl. A61K 31/506; C07D 471/04; A61K 31/437; A61P 35/00; A61P 13/12; A61P 25/28

(52) U.S. Cl. 514/275; 546/113; 514/300; 544/331

(57) ABSTRACT

The invention relates to pyrrolo[2,3-b]pyridine compounds and azaindole compounds used for the synthesis thereof. The invention also relates to methods for the production thereof and the uses thereof. Said novel pyrrolo[2,3-b]pyridine compounds according to the invention have great antiproliferative, apoptotic, and neuroprotective activities. The invention particularly applies to the pharmaceutical field.

The invention relates to pyrrolo[2,3-b]pyridine compounds and to azaindole compounds of use in the synthesis of these pyrrolo[2,3-b]pyridine compounds. It also relates to a process for the manufacture of these pyrrolo[2,3-b]pyridine compounds and to the use of these pyrrolo[2,3-b]pyridine compounds.

The phosphorylation of proteins is the mechanism most generally used by the cell for adjusting the activity of its structural proteins and of its enzymes. The phosphorylation of serine, threonine or tyrosine residues is catalyzed by a huge family of enzymes, the protein kinases. There is no important physiological event which does not involve modifications to the phosphorylation of proteins. Likewise, the very great majority of human pathologies involve anomalies of phosphorylation, often associated with anomalies in the regulation of certain protein kinases.

Increasing efforts have been directed in research in the last decade towards the optimization and the therapeutic evaluation of pharmacological inhibitors, with low molecular weights, of numerous protein kinases.

Currently, approximately 60 kinase inhibitors are in clinical evaluation against cancers, inflammation, diabetes and neurodegenerative diseases.

Among the 518 human kinases, cyclin-dependent kinases (CDKs) are attracting considerable interest due to their involvement in numerous essential physiological processes and many human diseases, especially cancers, polycystic kidney disease and neurodegenerative diseases, such as Alzheimer's disease, Parkinson's disease and strokes.

Consequently, numerous pharmacological inhibitors of CDKs have been described and demonstrated as having promising antitumor and/or antiproliferative and/or neuroprotective activities.

Some, such as flavopiridol, R-roscovitine and SNS-032, are in clinical evaluation as anticancer medicaments.

All the CDK inhibitors identified to date act by competing with ATP for bonding to the catalytic site of their kinase target. Many have been cocrystallized with a CDK target. The selectivity of these pharmacological inhibitors is the subject of intensive research using a great variety of methods, such as research of selectivity over a huge sample of kinases, affinity chromatography with regard to an immobilized inhibitor and the triple hybrid technique in the yeast.

While some kinase inhibitors are rather unselective, such as staurosporine, many exhibit a definite specificity profile. However, all inhibit several kinases.

Some also target unexpected nonprotein kinases. These multitarget inhibitors can have an appropriate medical use as they are less liable to bring about a phenomenon of resistance.

The 518 human kinases include the family of the DYRKs.

The gene of the protein kinase DYRK1A is located in a highly specific region of chromosome 21, the Down's syndrome critical region, which covers approximately twenty genes responsible for the trisomic phenotype. Many arguments support the hypothesis of an essential contribution of the overexpression, even modest (×1.5), of DYRK1A in the abnormal development of the brain observed during trisomy 21. Moreover, DYRK1A also appears to be strongly implicated in Alzheimer's disease (which furthermore appears in sufferers from Down's syndrome in a systematic and early fashion from the age of about forty). DYRK1A belongs to a small family of kinases comprising 5 members (DYRK1a, 1B, 2, 3 and 4). DYRK1A acts as priming kinase for GSK-3; it phosphorylates proteins of Alzheimer's disease, such as Tau and CRMP. The joint inhibition of CDKs, GSK-3, CK1 and DYRK might constitute a major advantage in the treatment of neurodegenerative diseases.

Meridianins, a family of 3-(2-aminopyrimidinyl)indoles, have recently been identified as promising kinase-inhibiting structures. Meridianins are natural products initially extracted from Aplidium meridianum, an ascidian from the South Atlantic. Meridianin derivatives have been synthesized by various groups of researchers. Although some meridianins inhibit various kinases, such as CDKs, synthase kinase-3 (GSK-3), cyclic nucleotide-dependent kinases and casein kinases 1 (CK1), they exhibit significant but modest antiproliferative effects.

Meridianins share a degree of structural similarity with variolins, another family of natural marine compounds comprising a central pyridopyrrolopyrimidine ring system substituted by a 2-aminopyrimidine ring. Variolins were initially extracted from Kirkpatrielda variolosa, a rare and difficult to access sponge from the Antarctic. They were subsequently synthesized. Variolin B and deoxyvariolin B (PM01218) exhibit a powerful cytotoxicity against several human cancer cell lines. Recently, variolin B and deoxyvariolin B have been reported as inhibiting CDKs.

Variolin analogs have formed the subject of intensive research.

These variolin analogs will be referred to hereinafter as meriolins. They are pyrrolo[2,3-b]pyridine compounds.

Thus, patent application WO 2006/050076 discloses numerous fused pyrrolyl compounds substituted by a pyrimidinyl ring of use in the treatment of disorders related to kinases.

Although numerous meriolin compounds and their synthesis processes are described in this document, no disclosure is made, however, of their pharmacological properties and in particular of any antiproliferative activity associated or not with inhibition with regard to cyclin-dependent kinases.

Patent application WO 2006/124863 also discloses numerous meriolin compounds presented as being able to be used to treat or prevent diseases or disorders associated with an abnormal or deregulated kinase activity, more particularly the diseases or disorders which involve abnormal activation, in particular, of CDKs.

However, no study of the activity of these compounds is disclosed in this document.

Patent application WO 2005/095400 discloses a very large number of azaindole compounds which are presented as protein kinase inhibitors.

However, once again, besides the fact that this patent application covers a very large number of compounds, no result demonstrating the effective activity of these compounds is disclosed.

The document “Synthesis of Polyheterocyclic Nitrogen-Containing Marine Natural Products”, Monatsh. Chem., 2004, 135, 615-627, discloses meriolins, including the meriolin subsequently referred to as meriolin 12, in which the substituent on the pyrimidine ring is an —SMe radical in the ² position, of formula:

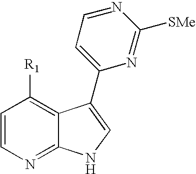

However, this document teaches that these compounds have no significant antitumor activity.

The document “Synthesis of the Indole Alkaloids Meridianins from the Tunicate Aplidium meridianum”, Tetrahedron, 2001, 57, 2355-2363, discloses a meriolin, referred to in the continuation of the text as meriolin 1, comprising an NH₂ substituent on the pyrimidine ring, of formula:

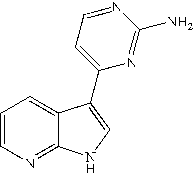

However, no result of tests of biological activity of this molecule is described.

The document “Concise Synthesis of Meridianins by Carbonylative Alkynylation and a Four-Component Pyrimidine Synthesis”, Angew. Chem. Int. Ed., 2005, 44, 6951-6956, also discloses meriolin 1 while indicating that this compound inhibits the kinases tested, that is to say hSGK1, Tie-2, VEGFR2/KDR, PDGF-receptor β kinase, Meek-BE kinase and IGF1 tyrosine kinase, at micromolar and even nanomolar levels.

More specifically, this compound, which will be referred to hereinafter as meriolin 1, has an IC₅₀ value of 2.4 μM for the protein kinase hSGK1.

Thus, in the prior art, meriolin compounds are described in a general fashion, in connection with their synthesis processes, but no specific biological activity of these compounds is disclosed.

In point of fact, it has now been discovered that a specific series of these meriolin compounds exhibits powerful inhibiting activities with regard to CDK kinases, especially CDK9, and other kinases, such as GSK-3 and CK1, and that they are antiproliferative and proapoptotic agents in cell cultures, due to their specific structure.

In particular, a structure/activity relationship has been demonstrated for this family of inhibitors acting in the ATP-binding pocket of kinases. The proapoptotic effectiveness of these meriolins is best demonstrated by their CDK9-inhibiting activity.

Thus, the invention relates to the compounds of following formula I:

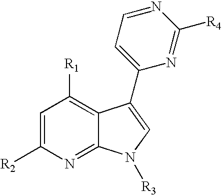

in which:
R₁ is chosen from a halogen or a substituted or unsubstituted C₁-C₁₀ alkyl group, a C₅-C₈ (C₁-C₁₀ alkyl)cycloalkyl group, a C₆-C₁₈ (C₁-C₁₀ alkyl)aryl group, an aromatic or nonaromatic C₅-C₁₂ (C₁-C₁₀ alkyl)heterocyclyl group comprising from one to three heteroatoms, a substituted or unsubstituted C₁-C₁₀ alkoxy group, a C₁-C₁₀ fluoroalkoxy group, a C₁-C₁₀ (C₁-C₁₀ alkoxy)alkoxy group, a C₅-C₈ cycloalkoxy group, a C₅-C₈ (C₁-C₁₀ alkoxy)cycloalkyl group, a C₆-C₁₈ (C₁-C₁₀ alkoxy)aryl group, an aromatic or nonaromatic C₅-C₁₂ (C₁-C₁₀ alkoxy)heterocyclyl group comprising from one to three heteroatoms, a substituted or unsubstituted. C₂-C₁₀ alkenyl group, a C₅-C₈ (C₂-C₁₀ alkenyl)cycloalkyl group, a C₆-C₁₈ (C₂-C₁₀ alkenyl)aryl group, an aromatic or nonaromatic C₅-C₁₂ (C₂-C₁₀ alkenyl)heterocyclyl group comprising from one to three heteroatoms, a substituted or unsubstituted C₂-C₁₀ alkynyl group, a C₅-C₈ (C₂-C₁₀ alkynyl)cycloalkyl group, a C₆-C₁₈ (C₂-C₁₀ alkynyl)aryl group, an aromatic or nonaromatic C₅-C₁₂ (C₂-C₁₀ alkynyl)heterocyclyl group comprising from one to three heteroatoms, an —OH group, an —OCORₐ group, a —CN group, an —NO₂ group, an —SRₐ group, an —NRₐR_{b} group, an —NHCORₐ group, an —NHSO₂Rₐ group, an —NHSO₂Rₐ group, an —NHCONRₐR_{b} group, an —NHCO₂Rₐ group, a phenyl group, a C₆-C₁₈ aryl group or an aromatic or nonaromatic C₅-C₁₂ heterocyclyl group comprising from one to three heteroatoms,
R₂ represents a hydrogen or halogen atom or, independently of R₁, a group as defined for R₁,
R₃ represents H or an —SO₂Rₐ, or —CORₐ or C₁-C₁₀ alkyl group,
R₄ represents a hydrogen atom or an NH₂ group,
Rₐ and R_{b} represent, each independently of one another, a hydrogen atom or an optionally substituted group chosen from a C₁-C₁₀ alkyl, a C₂-C₁₀ alkenyl, a C₂-C₁₀ alkynyl, a C₅-C₈ cycloalkyl, a C₁-C₁₀ (C₅-C₈ cycloalkyl)alkyl, a C₂-C₁₀ (C₅-C₈ cycloalkyl)alkenyl, a C₂-C₁₀ (C₅-C₈ cycloalkyl)alkynyl, a C₁-C₁₀ (C₅-C₁₂ heterocycle)alkyl, a C₂-C₁₀ (C₅-C₁₂ heterocycloalkyl)alkenyl or a C₂-C₁₀ (C₅-C₁₂ heterocycloalkyl)alkynyl or else Rₐ and R_{b} are bonded together to form, with the nitrogen atom to which they are bonded, an optionally substituted heterocycle chosen from a pyrrolidinyl, piperidinyl, piperazinyl and morpholinyl group, and the pharmaceutically acceptable salts of these compounds of formula I.

Preferably, in the compounds of formula
R₁ is chosen from a halogen or a C₁-C₁₀ alkyl, unsubstituted or substituted C₁-C₁₀ alkoxy, C₁-C₁₀ fluoroalkoxy, C₁-C₁₀ (C₁-C₁₀ alkoxy) alkoxy, C₅-C₈ cycloalkoxy, —OH, —OCORₐ, —CN, —NO₂, —SRₐ, —NRₐR_{b}, —NHCORₐ, —NHSO₂Rₐ, —NHCONRₐR_{b}, —NHCO₂Rₐ, phenyl, aryl or heteroaryl group,
R₂ represents a hydrogen atom or, independently of R₁, a halogen or a group as defined for R₁,
R₃ represents H or an —SO₂Rₐ, or —CORₐ or alkyl group,
R₄ represents a hydrogen atom or an NH₂ group,
Rₐ and R_{b} represent, each independently of one another, a hydrogen atom or an optionally substituted group chosen from alkyl, alkenyl, alkynyl, cycloalkyl, cycloalkylalkyl, cycloalkylalkenyl, cycloalkylalkynyl, heterocycloalkyl, heterocycloalkylalkyl or heterocycloalkylalkenyl groups or else Rₐ and R_{b} are bonded together to form, with the nitrogen atom to which they are bonded, an optionally substituted heterocycle chosen from a pyrrolidinyl, piperidinyl, piperazinyl or morpholinyl group.

This is because it has now been discovered that, for the meriolins to have an antiproliferative and/or apoptotic activity with regard to the cells, R₃ has to be H or an —SO₂Rₐ or —CORₐ or alkyl group.

Preferably, in the formula R₃ is H or an alkyl group.

Most preferably, R₃ is H.

It has also been discovered that the nature of the R₁ substituent on the pyridine ring is important for the cyclin-dependent kinase inhibitory activity, that is to say for the antiproliferative activity and/or for the apoptotic activity of the compounds of the invention and thus for their in particular antitumor activity.

More preferably, R₁ is chosen from an OH, Cl, methoxy, ethoxy, propoxy, butyloxy, isopropoxy, benzyloxy, cyclohexylmethoxy, cyclohexyloxy, 2-propylethynyl, 2-butylethynyl, 2-cyclohexylethynyl, pheneth-1-ynyl, phenyl, pentyl or phenylethyl group.

Most preferably, R₁ is chosen from the group formed by a methoxy, ethoxy, propoxy, isopropoxy, benzyloxy, cyclohexylmethyloxy, cyclohexyloxy, 2-propylethynyl, pentyl, phenyl and phenylethyl group.

This is because the tests carried out with the meriolins of the invention on five SH-SY5Y and HEK293 neuroblastoma cell lines, in comparison with the effect of variolin B, meriolin 1 and meriolin 12 described in the prior art, demonstrate a particularly powerful effect of the meriolins of the invention and in particular of the meriolins referred to hereinafter as meriolins 3, 4, 5, 6, 15, 16, 17, 18, 19, 22 and 23.

The effect of the tested compounds on the protein kinases CK1 and CDK9 demonstrates their exceptional antiproliferative effect resulting in cell death, in particular of tumor cells, and the effect of the tested meriolins on the protein kinases CDK5, GSK3 and CDK1 demonstrates their neuroprotective effect, as will be shown in the examples which follow.

The compounds of the invention were obtained by four processes which all use, as starting compounds, the compounds of following formula II:

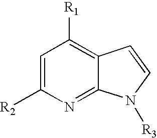

In these compounds, the R₁, R₂ and R₃ substituents are those defined for the compounds of formula I as being preferred.

These compounds make it possible to obtain the meriolins of the invention in 1 to 5 stages.

Thus, the meriolins of the invention can be used as medicament, in particular for all the disorders related to an abnormal activity of cyclin-dependent kinases. In particular, the meriolins of the invention can be used in the manufacture of a medicament for the treatment of disorders related to an abnormal proliferation of cancer or noncancer cells or as neuroprotector, that is to say for treating in particular tumors, neurodegenerative diseases, such as Alzheimer's disease and trisomy 21, leukemia, kidney diseases (glomerulonephritis, polycystic kidney disease), inflammation and type II diabetes. They can also be used for applications in combating parasites.

The invention will be better understood and other advantages and characteristics of the invention will become more clearly apparent in the light of the explanatory description which follows, which is made with reference to examples given purely by way of illustration and without implied limitation and to the figures, in which:

FIG. 1 represents the decrease in the survival of cells brought about by different concentrations of meriolins according to the invention, in comparison with that brought about by variolin B, at the same concentrations,

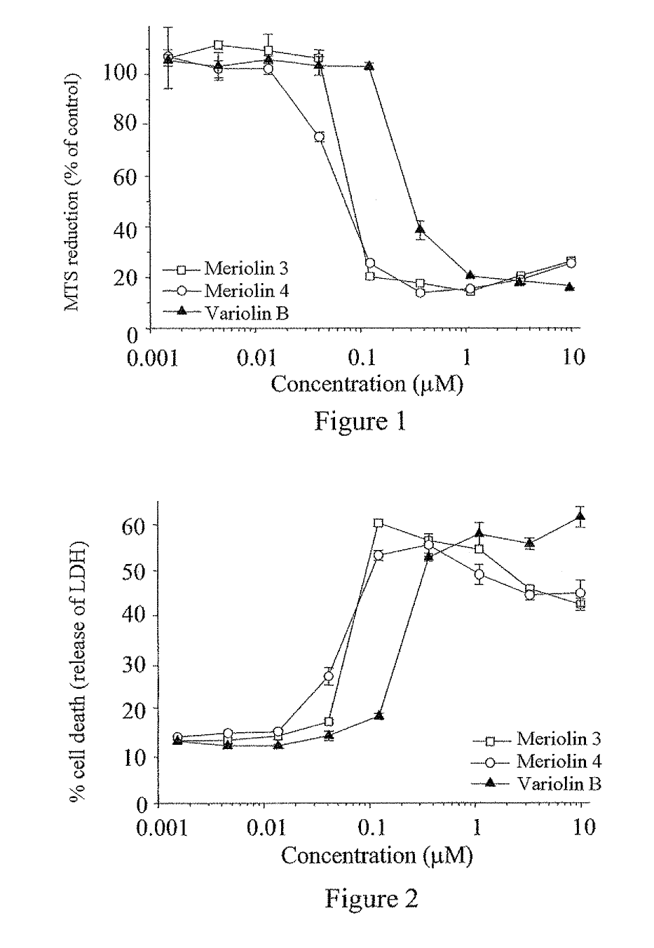

FIG. 2 represents the percentage of cell death brought about by different concentrations of meriolins according to the invention, in comparison with that brought about by variolin B, at the same concentrations, and

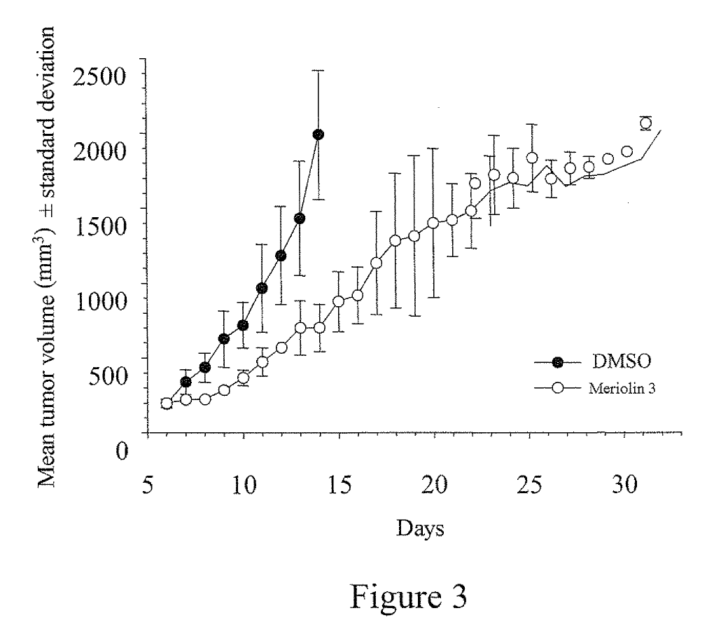

FIG. 3 represents the mean tumor volume of a tumor exposed to a meriolin according to the invention, in comparison with that of a tumor exposed to a control compound, that is to say an untreated tumor.

In the description which follows, the abbreviations used have the following meanings:

CDK: cyclin-dependent kinase,

CK1: casein kinase,

DYRK1A: dual-specificity tyrosine phosphorylation activated kinase,

FCS: fetal calf serum,

GSK3: glycogen synthase kinase-3,

LDH: lactate dehydrogenase,

MTS: 3-(4,5-dimethylthiazol-2-yl)-5-(3-carboxymethoxyphenyl)-2-(4-sulfophenyl)-2H-tetrazolium,

PBS: sulfate buffered saline solution,

DMSO: dimethyl sulfoxide.

### I) Synthesis of the Meriolins of the Invention

The meriolins of the invention were synthesized by the four processes represented diagrammatically in the following scheme 1:

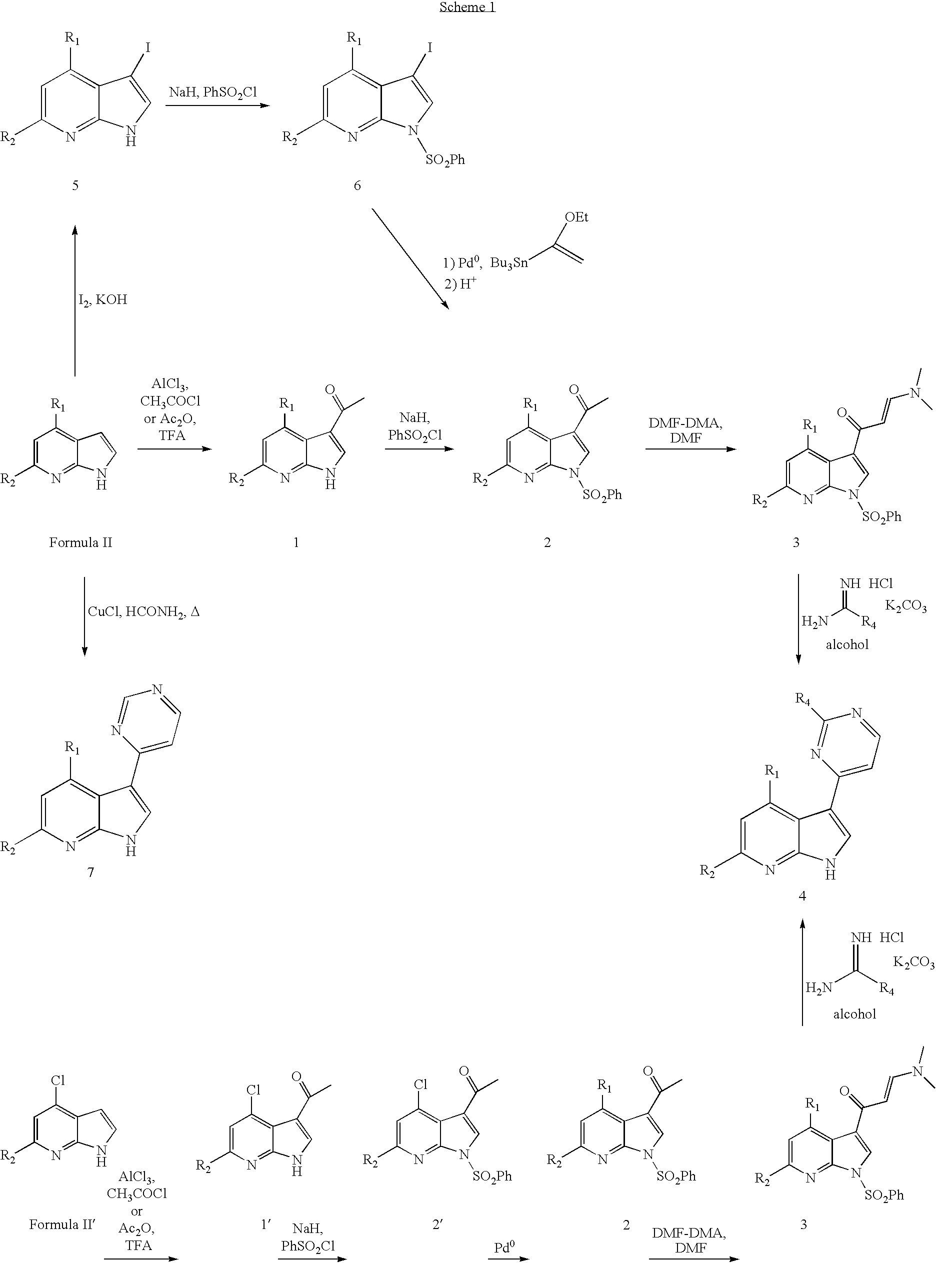

In scheme 1, the compound denoted Formula II′ is a compound of Formula II in which R₁ is chlorine.

### EXAMPLE 1

### Synthesis of the Compounds of Formula II

The starting compounds of formula II were synthesized in the following way.

The Mitsunobu reaction on the 4-hydroxy-7-azaindole derivative (J. Heterocyclic Chem., 1989, 26, 317-325) in the presence of various alcohols results in the production of the 7-azaindoles O-alkylated in the 4 position.

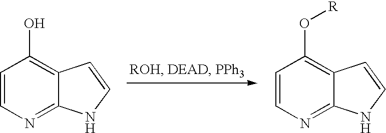

### 4-Ethoxy-1H-pyrrolo[2,3-b]pyridine

Diethyl azodicarboxylate (DEAD) (520 μl, 3.3 mmol) is added dropwise, at ambient temperature and under an inert atmosphere, to a solution of PPh₃ (1.04 g, 3.96 mmol) in anhydrous THF (13 ml). This solution is transferred via a tube, under an inert atmosphere, into a round-bottomed flask containing a solution of 4-hydroxy-7-azaindole (222 mg, 1.65 mmol) and ethanol (115 μl, 1.98 mmol) in anhydrous tetrahydrofuran (THE) (41 ml). The solution is stirred at ambient temperature for 2 h. The solvent is evaporated. The residue obtained is purified with a chromatography column (eluent: CH₂Cl₂/MeOH 98:2) to give the desired compound (214 mg, 80%). Solid; M.p.=176-178° C. (Et₂O); ¹H NMR (300 MHz, CDCl₃) δ 1.52 (t, 3H, J=7.1 Hz, CH₃), 4.26 (q, 2H, J=7.1 Hz, CH₂), 6.53 (d, 1H, J=5.6 Hz, Hₐᵣₒₘ), 6.59 (broad s, 1H, Hₐᵣₒₘ), 7.18 (broad s, 1H, Hₐᵣₒₘ), 8.18 (d, 1H, J=5.6 Hz, Hₐᵣₒₘ), 9.64 (broad s, 1H, NH); MS (SI) m/z 163 (M+H⁺).

### 4-Propoxy-1H-pyrrolo[2,3-b]pyridine

4-Propoxy-1H-pyrrolo[2,3-b]pyridine is obtained, according to the procedure described for the preparation of 4-ethoxy-1H-pyrrolo[2,3-b]pyridine, with a yield of 78% from 4-hydroxy-7-azaindole and propanol. Solid; M.p.=189-191° C. (Et₂O); ¹H NMR (300 MHz, CDCl₃) δ 1.10 (t, 3H, J=7.4 Hz, CH₃), 1.88-1.99 (m, 2H, CH₂), 4.20 (t, 2H, J=6.6 Hz, CH₂), 6.59 (d, 1H, J=5.8 Hz, Hₐᵣₒₘ), 6.63 (d, 1H, J=3.6 Hz, Hₐᵣₒₘ), 7.21 (d, 1H, J=3.6 Hz, Hₐᵣₒₘ), 8.15 (d, 1H, J=5.8 Hz, Hₐᵣₒₘ); MS (SI) m/z 177 (M+H⁺).

### 4-(1-Methylethoxy)-1H-pyrrolo[2,3-b]pyridine

4-(1-Methylethoxy)-1H-pyrrolo[2,3-b]pyridine is obtained, according to the procedure described for the preparation of 4-ethoxy-1H-pyrrolo[2,3-b]pyridine, with a yield of 76% from 4-hydroxy-7-azaindole and 2-propanol. Solid; M.p.=182-184° C. (Et₂O); ¹H NMR (300 MHz, CDCl₃) δ 1.44 (d, 6H, J=6.0 Hz, 2 CH₃), 4.82 (hept, 1H, J=6.0 Hz, CH), 6.52 (d, 1H, J=5.7 Hz, Hₐᵣₒₘ), 6.57 (d, 1H, J=3.6 Hz, Hₐᵣₒₘ), 7.19 (d, 1H, J=3.6 Hz, Hₐᵣₒₘ), 8.17 (d, 1H, J=5.7 Hz, Hₐᵣₒₘ), 10.70 (broad s, 1H, NH); MS (SI) m/z 177 (M+H⁺).

### 4-(Benzyloxy)-1H-pyrrolo[2,3-b]pyridine

4-(Benzyloxy)-1H-pyrrolo[2,3-b]pyridine is obtained, according to the procedure described for the preparation of 4-ethoxy-1H-pyrrolo[2,3-b]pyridine, with a yield of 61% from 4-hydroxy-7-azaindole and benzyl alcohol. Solid; ¹H NMR (300 MHz, CDCl₃) δ 5.46 (s, 2H, CH₂), 6.78 (d, 1H, J=3.6 Hz, Hₐᵣₒₘ), 6.85 (d, 1H, J=6.9 Hz, Hₐᵣₒₘ), 7.35 (d, 1H, J=3.6 Hz, Hₐᵣₒₘ), 7.40-7.50 (m, 5H, Hₐᵣₒₘ), 8.10 (d, 1H, J=6.9 Hz, Hₐᵣₒₘ), 11.78 (broad s, 1H, NH). MS (SI): m/z 225 (M+H⁺).

### 4-(Cyclohexylmethoxy)-1H-pyrrolo[2,3-b]pyridine

4-(Cyclohexylmethoxy)-1H-pyrrolo[2,3-b]pyridine is obtained, according to the procedure described for the preparation of 4-ethoxy-1H-pyrrolo[2,3-b]pyridine, with a yield of 52% from 4-hydroxy-7-azaindole and cyclohexylmethanol. Solid; ¹H NMR (300 MHz, CDCl₃) δ 1.06-1.40 (m, 5H, CH₂), 1.71-1.95 (m, 6H, CH+CH₂), 3.99 (d, 2H, J=6.0 Hz, CH₂), 6.54 (d, 1H, J=5.6 Hz, Hₐᵣₒₘ), 6.60 (d, 1H, J=3.4 Hz, Hₐᵣₒₘ), 7.20 (d, 1H, J=3.4 Hz, Hₐᵣₒₘ), 8.18 (d, 1H, J=5.6 Hz, Hₐᵣₒₘ), 10.61 (broad s, 1H, NH). MS (SI): m/z 231 (M+H⁺).

### 4-(Cyclohexyl)-1H-pyrrolo[2,3-b]pyridine

4-(Cyclohexyl)-1H-pyrrolo[2,3-b]pyridine is obtained, according to the procedure described for the preparation of 4-ethoxy-1H-pyrrolo[2,3-b]pyridine, with a yield of 49% from 4-hydroxy-7-azaindole and cyclohexanol. Solid; ¹H NMR (300 MHz, CDCl₃): δ 1.30-1.48 (m, 3H, CH₂), 1.60-1.71 (m, 3H, CH₂), 1.85 (broad s, 2H, CH₂), 2.04 (broad s, 2H, CH₂), 4.54-4.61 (m, 1H, CH), 6.57 (d, 1H, J=5.8 Hz, Hₐᵣₒₘ), 6.60 (d, 1H, J=3.5 Hz, Hₐᵣₒₘ), 7.20 (d, 1H, J=3.5 Hz, Hₐᵣₒₘ), 8.14 (d, 1H, J=5.8 Hz, Hₐᵣₒₘ), 10.33 (broad s, 1H, NH); MS (SI): m/z 217 (M+H⁺).

The 7-azaindoles of formula II O-alkylated in the 4 position (C₁-C₁₀ alkoxy, C₁-C₁₀ fluoroalkoxy, substituted C₁-C₁₀ alkoxy, C₅-C₈ cycloalkoxy, benzyloxy, aryloxy, heteroaryloxy or heteroarylalkyloxy) were prepared according to this method.

Thus, the compounds of formula II according to the invention were obtained.

Starting from the compounds of formula II, the first process for producing the meriolins of the invention consists in carrying out an acylation reaction, in the presence of aluminum chloride (AlCl₃), of CH₃COCl, as described in J. Org. Chem., 2002, 67, 6226-6227, or of Ac₂O and trifluoroacetic acid, on the compounds of formula II substituted in the 4 position and/or in the 6 position, which results in the preparation of the compound denoted 1 in scheme 1.

Subsequently, a benzenesulfonylation reaction on the N-1 indole nitrogen of the derivatives 1 is carried out in a basic medium in the presence of benzenesulfonyl chloride to give the compounds denoted 2 in scheme 1.

The enaninones denoted 3 in scheme I are then obtained by reaction of the compounds 2 with DMF-DMA in DMF according to the method described in Tetrahedron, 2001, 57, 2355-2363.

The substituted pyrimidine ring is for its part formed by treating the compound 3 in the presence of guanidinium hydrochloride or its derivatives.

The final compound, denoted 4 in scheme 1 (not protected on the N-1 indole nitrogen), is obtained with a good yield.

The second process for producing the meriolins of the invention differs from process 1 in the stage for producing the compounds 2. In this second process, the compounds of formula II substituted in the 4 position and/or in the 6 position are iodated in the 3 position in the presence of iodine in a basic medium to give the derivatives denoted 5 in scheme 1. The benzenesulfonylation reaction on the N-1 indole nitrogen of the derivatives 5 gives the compounds denoted 6 in scheme 1. The latter are used in a coupling reaction catalyzed by palladium (Stille reaction) in the presence of tri-(n-butyl(1-ethoxyvinyl)stannane to give the compounds 2.

Subsequently, the compounds 4 are obtained from these compounds 2 by the same stages as for the first process described.

The third process makes it possible to obtain the meriolins in which the R₄ substituent on the pyrimidine ring is H. The compound denoted 7 in scheme 1 is obtained directly from the compounds of formula II by treatment with CuO and formamide.

The fourth process for producing the meriolins of the invention differs from processes 1 and 2 in the stage for producing the compounds 2. In this fourth process, the 4 position of the 7-azaindole is functionalized starting from 7-azaindoles of formula IP substituted by a chlorine in the 4 position. This stage is carried out by reactions catalyzed by palladium (Sonogashira reaction, Stille reaction, Suzuki-Myaura reaction, Heck reaction, and the like) to result in the compounds 2.

Subsequently, the compounds 4 are obtained from these compounds 2 by the same stages as for the first process described.

### EXAMPLE 2

### Synthesis of 3-[(2-amino)pyrimidin-4-yl]-4-chloro-1H-pyrrolo[2,3-b]pyridine (4a): meriolin 10.

### a) 3-Acetyl-4-chloro-1H-pyrrolo[2,3-b]pyridine (1a)

Method A: Aluminum chloride (1.70 g, 12.78 mmol) is added, at ambient temperature and under an inert atmosphere, to a solution of 4-chloro-7-azaindole (0.32 g, 2.13 mmol) in anhydrous CH₂Cl₂ (15 ml). The solution is stirred at ambient temperature for 90 min. Acetyl chloride (0.91 ml, 12.78 mmol) is added dropwise at ambient temperature. The final solution is stirred at ambient temperature for 5 days. After addition of MeOH, the solvents are evaporated. The residue obtained is taken up in a 1N NaOH and AcOEt mixture (200 ml 1:1) and then the two phases are separated. The organic phase collected is dried over MgSO₄ and then evaporated. The solid obtained is purified with a chromatography column (eluent: AcOEt) to give the compound 1a (320 mg, 77%).

Method B: A solution of 4-chloro-7-azaindole (90 mg, 0.59 mmol), acetic anhydride (0.13 ml, 1.8 mmol) and trifluoroacetic acid (5 ml) is heated at reflux for 8 h. After cooling, a saturated Na₂CO₃ solution is added in order to neutralize the medium (pH=7-8). After adding AcOEt, stirring and settling, the phases are separated. The organic phase is dried over MgSO₄ and then evaporated. The residue is purified with a chromatography column (eluent: AcOEt) to give 1a (98 mg, 85%). M.p.>210° C. (MeOH); ¹H NMR (300 MHz, CDCl₃) δ 2.63 (s, 3H, CH₃), 7.41 (d, 1H, J=5.6 Hz, Hₐᵣₒₘ), 8.12 (s, 1H, Hₐᵣₒₘ), 8.27 (d, 1H, J=5.6 Hz, Hₐᵣₒₘ); MS (SI) m/z 195 (M+H⁺).

### b) 3-Acetyl-1-benzenesulfonyl-4-chloro-1H-pyrrolo[2,3-b]pyridine (2a)

Sodium hydride (26 mg, 0.64 mmol, 60% in oil) is added in small portions to a solution of compound 1a (125 mg, 0.64 mmol) in anhydrous THF (20 ml) at 0° C. The solution is stirred at 0° C. for 45 min and then benzenesulfonyl chloride (0.11 ml, 0.83 mmol) is added to the reaction mixture. The final solution is stirred at ambient temperature for 4 h. The addition of H₂O is carried out at 0° C. and then the solvents are evaporated. The residue obtained is taken up in an H₂O and AcOEt mixture (50 ml, 1:1) and then the two phases are separated. The organic phase collected is dried over MgSO₄ and then evaporated. The solid obtained is purified with a chromatography column (eluent: petroleum ether/AcOEt 8:2) to give the compound 2a (200 mg, 93%). M.p.=160-162° C. (CH₂Cl₂/pentane); ¹H NMR (300 MHz, CDCl₃) δ 2.63 (s, 3H, CH₃), 7.30 (d, 1H, J=5.2 Hz, Hₐᵣₒₘ), 7.54 (broad t, 2H, J=7.4 Hz, Hₐᵣₒₘ), 7.66 (broad t, 1H, J=7.4 Hz, Hₐᵣₒₘ), 8.25 (broad d, 2H, J=7.4 Hz, Hₐᵣₒₘ), 8.32 (d, 1H, J=5.2 Hz, Hₐᵣₒₘ) 8.34 (s, 1H, Hₐᵣₒₘ); MS (SI) m/z 335 (M+H⁺).

### c) 1-(1-Benzenesulfonyl-4-chloro-1H-pyrrolo[2,3-b]pyridin-3-yl)-3,3-dimethylaminopropenone (3a)

A solution of 2a (450 mg, 1.34 mmol) and DMF-DMA (1.07 ml, 8.4 mmol) in anhydrous DMF (15 ml) is stirred at 90° C. for 8 h under an inert atmosphere. After cooling, the solvent is evaporated. The residue obtained is taken up in an H₂O and AcOEt mixture (50 ml, 1:1) and then the two phases are separated. The organic phase collected is dried over MgSO₄ and then evaporated. The solid obtained is purified with a chromatography column (eluent: AcOEt) to give the compound 3a (310 mg, 60%). M.p.=139-141° C. (MeOH); ¹H NMR (300 MHz, CDCl₃) δ 2.93 (broad s, 3H, CH₃), 3.15 (broad s, 3H, CH₃), 5.49 (d, 1H, J=12.6 Hz, ═CH), 7.23 (d, 1.14, J=5.3 Hz, Hₐᵣₒₘ), 7.49 (t, 2H, J=7.4 Hz, Hₐᵣₒₘ), 7.47-7.65 (m, 2H, Hₐᵣₒₘ+═CH), 8.00 (s, 1H, Hₐᵣₒₘ), 8.19 (d, 2H, J=7.7 Hz, Hₐᵣₒₘ), 8.29 (d, 1H, J=5.3 Hz, Hₐᵣₒₘ); MS (SI) m/z 390 (M+H⁺).

### d) 3-[(2-Amino)pyrimidin-4-yl]-4-chloro-1H-pyrrolo[2,3-b]pyridine (4a)

A solution of 3a (140 mg, 0.36 mmol), guanidinium hydrochloride (52 mg, 0.54 mmol) and K₂CO₃ (106 mg, 0.76 mmol) in 2-methoxyethanol (5 ml) is heated at 100-110° C. for 36 h. After cooling, the solution is run quickly into water. The final solution is extracted with AcOEt (2×). The combined organic phase is dried over MgSO₄ and then evaporated. The residue is purified with a chromatography column (CH₂Cl₂/MeOH 95:5) to give 4a (45 mg, 51%). M.p.>210° C. (MeOH); ¹H NMR (300 MHz, d₆-DMSO) δ 6.48 (broad s, 2H, NH₂), 6.85 (d, 1H, J=5.3 Hz, Hₐᵣₒₘ), 7.26 (d, 1H, J=5.3 Hz, Hₐᵣₒₘ), 7.94 (s, 1H, Hₐᵣₒₘ), 8.20 (d, 1H, J=5.3 Hz, Hₐᵣₒₘ), 8.22 (d, 1H, J=5.3 Hz, Hₐᵣₒₘ), 12.49 (broad s, 1H, NH); MS (SI) m/z 246 (M+H⁺).

### EXAMPLE 3

### Synthesis of 3-[(2-amino)pyrimidin-4-yl]-4-methoxy-1H-pyrrolo[2,3-b]pyridine (4b): meriolin 3.

### a) 3-Acetyl-4-methoxy-1H-pyrrolo[2,3-b]pyridine (1b)

The compound 1b is obtained, according to the procedure described for the preparation of 1a, with a yield of 55% from 4-methoxy-7-azaindole. M.p.>210° C. (MeOH); ¹H NMR (300 MHz, d₆-DMSO) δ 2.50 (s, 3H, CH₃), 3.92 (s, 3H, CH₃), 6.79 (d, 1H, J=5.6 Hz, Hₐᵣₒₘ), 8.11 (s, 1H, Hₐᵣₒₘ), 8.17 (d, 1H, J=5.6 Hz, Hₐᵣₒₘ), 12.36 (broad s, 1H, NH); MS (SI) m/z 191 (M+H⁺).

### b) 3-Acetyl-1-benzenesulfonyl-4-methoxy-1H-pyrrolo[2,3-b]pyridine (2b)

The compound 2b is obtained, according to the procedure described for the preparation of 2a, with a yield of 90% from 1b. M.p. 156-158° C. (CH₂Cl₂/pentane); ¹H NMR (300 MHz, CDCl₃) δ 2.63 (s, 3H, CH₃), 3.98 (s, 3H, CH₃), 6.73 (d, 1H, J=5.7 Hz, Hₐᵣₒₘ), 7.51 (broad t, 2H, J=7.4 Hz, Hₐᵣₒₘ), 7.62 (broad t, 1H, J=7.4 Hz, Hₐᵣₒₘ), 8.21 (s, 1H, Hₐᵣₒₘ), 8.24 (broad d, 2H, J=7.4 Hz, Hₐᵣₒₘ), 8.34 (d, 1H, J=5.7 Hz, Hₐᵣₒₘ); MS (SI) m/z 331 (M+H⁺).

### c) 1-(1-Benzenesulfonyl-4-methoxy-1H-pyrrolo[2,3-h]pyridin-3-yl)-3,3-dimethylaminopropenone (3b)

The compound 3b is obtained, according to the procedure described for the preparation of 3a, with a yield of 78% from 2b. M.p. 210-212° C. (MeOH); ¹H NMR (300 MHz, CDCl₃) δ 2.91 (broad s, 3H, CH₃), 3.12 (broad s, 3H, CH₃), 3.94 (s, 3H, CH₃), 5.62 (d, 1H, J=12.6 Hz, ═CH), 6.68 (d, 1H, J=5.7 Hz, Hₐᵣₒₘ), 7.47 (t, 2H, J=7.4 Hz, Hₐᵣₒₘ), 7.57 (t, 1H, J=7.4 Hz, Hₐᵣₒₘ), 7.65 (broad d, 1H, J=12.6 Hz, ═CH), 7.96 (s, 1H, Hₐᵣₒₘ), 8.18 (d, 2H, J=7.4 Hz, Hₐᵣₒₘ), 8.31 (d, 1H, J=5.7 Hz, Hₐᵣₒₘ); MS (SI) m/z 386 (M+H⁺).

### d) 3-[(2-Amino)pyrimidin-4-yl]-4-methoxy-1H-pyrrolo[2,3-b]pyridine (4b)

The compound 4b is obtained, according to the procedure described for the preparation of 4a, with a yield of 75% from 3b. M.p.>210° C. (MeOH); ¹H NMR (300 MHz, d₆-DMSO) δ 3.97 (s, 3H, CH₃), 6.32 (broad s, 2H, NH₂), 6.79 (d, 1H, J=5.5 Hz, Hₐᵣₒₘ), 7.27 (d, 1H, J=5.3 Hz, Hₐᵣₒₘ), 7.92 (s, 1H, Hₐᵣₒₘ), 8.16 (d, 1H, J=5.5 Hz, Hₐᵣₒₘ), 8.17 (d, 1H, J=5.3 Hz, Hₐᵣₒₘ), 12.13 (broad s, 1H, NH); MS (SI) m/z 242 (M+H⁺).

### EXAMPLE 4

### Synthesis of 3-[(2-amino)pyrimidin-4-yl]-4-ethoxy-1H-pyrrolo[2,3-b]pyridine (4c): meriolin 4.

### a) 3-Acetyl-4-ethoxy-1H-pyrrolo[2,3-b]pyridine (1c)

The compound 1c is obtained, according to the procedure described for the preparation of 1a, with a yield of 84% from 4-ethoxy-7-azaindole. M.p.>210° C. (MeOH); ¹H NMR (300 MHz, d₆-DMSO)δ 1.43 (t, 3H, J=7.2 Hz, CH₃), 2.54 (s, 3H, CH₃), 4.22 (q, 2H, J=7.2 Hz, CH₂), 6.78 (d, 1H, J=5.7 Hz, Hₐᵣₒₘ), 8.04 (s, 1Hₐᵣₒₘ), 8.14 (d, 1H, J=5.7 Hz, Hₐᵣₒₘ), 12.33 (broad s, 1H, NH); MS (SI) m/z 205 (M+H⁺).

### b) 3-Acetyl-1-benzenesulfonyl-4-ethoxy-1H-pyrrolo[2,3-b]pyridine (2c)

The compound 2c is obtained, according to the procedure described for the preparation of 2a, with a yield, of 68% from 1c. M.p.=151-153° C. (CH₂Cl₂/pentane); ¹H NMR (300 MHz, CDCl₃) δ 1.52 (t, 3H, J=7.2 Hz, CH₃), 2.65 (s, 3H, CH₃), 4.21 (q, 2H, J=7.2 Hz, CH₂), 6.70 (d, 1H, J=5.7 Hz, Hₐᵣₒₘ), 7.51 (broad t, 2H, J=7.4 Hz, Hₐᵣₒₘ), 7.61 (broad t, 1H, J=7.4 Hz, Hₐᵣₒₘ), 8.20 (s, 1H, Hₐᵣₒₘ), 8.23 (broad d, 2H, J=7.4 Hz, Hₐᵣₒₘ), 8.31 (d, 1H, J=5.7 Hz, Hₐᵣₒₘ); MS (SI) m/z 345 (M+H⁺).

### c) 1-(1-Benzenesulfonyl-4-ethoxy-1H-pyrrolo[2,3-b]pyridin-3-yl)-3,3-dimethylaminopropenone (3c)

The compound 3c is obtained, according to the procedure described for the preparation of 3a, with a yield of 68% from 2c. M.p.=187-189° C. (MeOH); ¹H NMR (300 MHz, CDCl₃) δ 1.44 (t, 3H, J=7.2 Hz, CH₃), 2.90 (broad s, 3H, CH₃), 3.11 (broad s, 3H, CH₃), 4.18 (q, 2H, J=7.2 Hz, CH₂), 5.60 (d, 1H, J=12.6 Hz, ═CH), 6.65 (d, 1H, J=5.8 Hz, Hₐᵣₒₘ), 7.46 (t, 2H, J=7.1 Hz, Hₐᵣₒₘ), 7.55-7.63 (m, 2H, Hₐᵣₒₘ+═CH), 7.94 (s, 1H, Hₐᵣₒₘ) 8.18 (d, 2H, J=7.7 Hz, Hₐᵣₒₘ), 8.29 (d, 1H, J=5.6 Hz, Hₐᵣₒₘ); MS (SI) m/z 400 (M+H⁺).

### d) 3-[(2-Amino)pyrimidin-4-0]-4-ethoxy-1H-pyrrolo[2,3-b]pyridine (4c)

The compound 4c is obtained, according to the procedure described for the preparation of 4a, with a yield of 63% from 3c. M.p.>210° C. (MeOH); ¹H NMR (300 MHz, d₆-DMSO) δ 1.45 (t, 3H, J=7.2 Hz, CH₃), 4.26 (q, 2H, J=7.2 Hz, CH₂), 6.33 (broad s, 2H, NH₂), 6.75 (d, 1H, J=5.5 Hz, Hₐᵣₒₘ), 7.36 (d, 1H, J=5.3 Hz, Hₐᵣₒₘ) 7.90 (s, 1H, Hₐᵣₒₘ), 8.13 (d, 1H, J=5.5 Hz, Hₐᵣₒₘ) 8.17 (d, 1H, J=5.3 Hz, Hₐᵣₒₘ) 12.09 (broad s, 1H, NH); MS (SI) m/z 256 (M+H⁺).

### EXAMPLE 5

### Synthesis of 3-[(2-amino)pyrimidin-4-yl]-4-propoxy-1H-pyrrolo[2,3-b]pyridine (4d): meriolin 5.

### a) 3-Acetyl-4-propoxy-1H-pyrrolo[2,3-b]pyridine (1d)

The compound 1d is obtained, according to the procedure described for the preparation of 1a, with a yield of 80% from 4-propoxy-7-azaindole. M.p.>210° C. (MeOH); ¹H NMR (300 MHz, d₆-DMSO) δ 1.07 (t, 3H, J=7.3 Hz, CH₃), 1.77-1.88 (m, 2H, CH₂), 2.53 (s, 3H, CH₃), 4.11 (t, 2H, J=6.3 Hz, CH₂), 6.78 (d, 1H, J=5.6 Hz, Hₐᵣₒₘ), 8.08 (s, 1H, Hₐᵣₒₘ), 8.14 (d, 1H, J=5.6 Hz, Hₐᵣₒₘ), 12.32 (broad s, 1H, NH); MS (SI) m/z 219 (M+H⁺).

### b) 3-Acetyl-1-benzenesulfonyl-4-propoxy-1H-pyrrolo[2,3-b]pyridine (2d)

The compound 2d is obtained, according to the procedure described for the preparation of 2a, with a yield of 70% from 1d. M.p. 119-121° C. (CH₂Cl₂/pentane); ¹H NMR (300 MHz, CDCl₃) δ 1.09 (t, 3H, J=7.3 Hz, CH₃), 1.87-1.96 (m, 2H, CH₂), 2.64 (s, 3H, CH₃), 4.09 (t, 2H, J=6.6 Hz, CH₂), 6.70 (d, 1H, J=5.7 Hz, Hₐᵣₒₘ), 7.51 (broad t, 2H, J=7.4 Hz, Hₐᵣₒₘ), 7.61 (broad t, 1H, J=7.4 Hz, Hₐᵣₒₘ), 8.19 (s, 1H, Hₐᵣₒₘ), 8.23 (broad d, 2H, J=7.4 Hz, Hₐᵣₒₘ), 8.31 (d, 1H, J=5.7 Hz, Hₐᵣₒₘ); MS (SI) m/z 359 (M+H⁺).

### c) 1-(1-Benzenesulfonyl-4-propoxy-1H-pyrrolo[2,3-b]pyridin-3-yl)-3,3-dimethylaminopropenone (3d)

The compound 3d is obtained, according to the procedure described for the preparation of 3a, with a yield of 69% from 2d. M.p.=102-104° C. (CH₂Cl₂/pentane); ¹H NMR (300 MHz, CDCl₃) δ 1.02 (t, 3H, J=7.3 Hz, CH₃), 1.77-1.89 (m, 2H, CH₂), 2.90 (broad s, 3H, CH₃), 3.10 (broad s, 3H, CH₃), 4.05 (q, 2H, J=6.4 Hz, CH₂), 5.59 (d, 1H, =12.6 Hz, ═CH), 6.64 (d, 1H, J=5.6 Hz, Hₐᵣₒₘ), 7.46 (t, 2H, J=7.4 Hz, Hₐᵣₒₘ), 7.56 (broad t, 1H, J=7.4 Hz, Hₐᵣₒₘ), 7.62 (broad d, 1H, J=12.6 Hz, ═CH), 7.91 (s, 1H, Hₐᵣₒₘ), 8.18 (d, 2H, J=7.4 Hz, Hₐᵣₒₘ), 8.28 (d, 1H, J=5.6 Hz, Hₐᵣₒₘ); MS (SI) m/z 414 (M+H⁺).

### d) 3-[(2-Amino)pyrimidin-4-yl]-4-propoxy-1H-pyrrolo[2,3-b]pyridine (4d)

The compound 4d is obtained, according to the procedure described for the preparation of 4a, with a yield of 60% from 3d. M.p.>210° C. (MeOH); ¹H NMR (300 MHz, d₆-DMSO) δ 1.01 (t, 3H, J=7.3 Hz, CH₃), 1.81-1.90 (m, 2H, CH₂), 4.16 (t, 2H, J=6.2 Hz, CH₂), 6.32 (broad s, 2H, NH₂), 6.76 (d, 1H, J=5.5 Hz, Hₐᵣₒₘ), 7.34 (d, 1H, J=5.3 Hz, Hₐᵣₒₘ), 7.90 (s, 1H, Hₐᵣₒₘ), 8.14 (d, 1H, J=5.5 Hz, Hₐᵣₒₘ), 8.16 (d, 1H, J=5.3 Hz, Hₐᵣₒₘ), 12.08 (s, 1H, NH); MS (SI) adz 270 (M+H⁺).

### EXAMPLE 6

### Synthesis of 3-[(2-amino)pyrimidin-4-yl]-4-(cyclohexylmethoxy)-1H-pyrrolo[2,3-b]pyridine (4e): meriolin 16

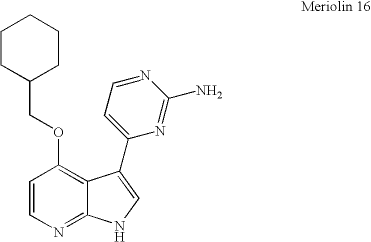

### a) 3-Acetyl-4-(cyclohexylmethoxy)-1H-pyrrolo[2,3-b]pyridine (1e)

The compound 1e is obtained, according to the procedure described for the preparation of 1a, with a yield of 99% from 4-cyclohexylmethoxy-7-azaindole. M.p.>210° C. (MeOH); ¹H NMR (300 MHz, d₆-DMSO) δ 1.08-1.34 (m, 5H, CH₂), 1.65-1.94 (m, 6H, CH₂), 2.51 (s, 3H, CH₃), 3.95 (d, 2H, J=6.0 Hz, CH₂), 6.76 (d, 1H, J=5.6 Hz, Hₐᵣₒₘ), 8.10 (s, 1H, Hₐᵣₒₘ), 8.13 (d, 1H, J=5.6 Hz, Hₐᵣₒₘ), 12.30 (broad s, 1H, NH); MS (SI) m/z 273 (M+H⁺).

### b) 3-Acetyl-1-benzenesulfonyl-4-(cyclohexylmethoxy)-1H-pyrrolo[2,3-b]pyridine (2e)

The compound 2e is obtained, according to the procedure described for the preparation of 2a, with a yield of 87% from 1e. M.p.=138-140° C. (MeOH); ¹H NMR (300 MHz, CDCl₃) δ 1.00-1.34 (m, 5H, CH₂), 1.65-1.88 (m, 6H, CH₂), 2.61 (s, 3H, CH₃), 3.89 (d, 2H, J=6.0 Hz, CH₂), 6.68 (d, 1H, J=5.8 Hz, Hₐᵣₒₘ), 7.45 (t, 1H, J=7.9 Hz, Hₐᵣₒₘ), 7.57 (t, 2H, J=7.4 Hz, Hₐᵣₒₘ), 8.16 (s, 1H, Hₐᵣₒₘ), 8.20 (d, 2H, J=7.5 Hz, Hₐᵣₒₘ), 8.27 (d, 1H, J=5.6 Hz, Hₐᵣₒₘ); MS (SI) m/z 413 (M+H⁺).

### c) 1-(1-Benzenesulfonyl)-4-(cyclohexylmethoxy)-1H-pyrrolo[2,3-b]pyridin-3-yl)-3,3-dimethylaminopropenone (3e)

The compound 3e is obtained, according to the procedure described for the preparation of 3a, with a yield of 73% from 2e. M.p.=102-104° C. (MeOH); ¹H NMR (300 MHz, CDCl₃) δ 0.97-1.25 (m, 5H, CH₂), 1.65-1.79 (m, 6H, CH+CH₂), 2.83 (broad s, 3H, CH₃), 3.04 (broad s, 3H, CH₃), 3.82 (d, 2H, J=5.6 Hz, CH₂), 5.48 (d, 1H, J=12.6 Hz, ═CH), 6.60 (d, 1H, J=5.6 Hz, Hₐᵣₒₘ), 7.41 (t, 2H, J=7.5 Hz, Hₐᵣₒₘ), 7.49-7.53 (m, 2H, ═CH+Hₐᵣₒₘ), 7.82 (s, 1H, Hₐᵣₒₘ), 8.13 (broad d, 2H, J=8.1 Hz, Hₐᵣₒₘ), 8.22 (d, 1H, =5.6 Hz, Hₐᵣₒₘ); MS (SI) m/z 468 (M+H⁺).

### d) 3-[(2-Amino)pyrimidin-4-yl]-(4-cyclohexylmethoxy)-1H-pyrrolo[2,3-b]pyridine (4e)

The compound 4e is obtained, according to the procedure described for the preparation of 4a, with a yield of 72% from 3e. M.p.>210° C. (MeOH); ¹H NMR (300 MHz, d₆-DMSO) δ 1.07-1.30 (m, 5H, CH₂), 1.65-1.83 (m, 6H, CH+CH₂), 4.01 (d, 2H, J=5.7 Hz, CH₂), 6.34 (broad s, 2H, NH₂), 6.75 (d, 1H, J=5.6 Hz, Hₐᵣₒₘ), 7.32 (d, 1H, J=5.3 Hz, Hₐᵣₒₘ), 7.87 (s, 1H, Hₐᵣₒₘ), 8.11-8.14 (m, 2H, Hₐᵣₒₘ), 12.08 (broad s, 1H, NH); MS (SI) m/z 324 (M+H⁺).

### EXAMPLE 7

### Synthesis of 3-[(2-amino)pyrimidin-4-yl]-4-phenyl-1H-pyrrolo[2,3-b]pyridine (4f): meriolin 23

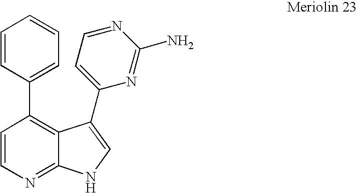

### a) 3-Acetyl-4-phenyl-1H-pyrrolo[2,3-b]pyridine (1f)

The compound 1f is obtained, according to the procedure described for the preparation of 1a, with a yield of 74% from 4-phenyl-7-azaindole (Synlett, 2001, 609). M.p.>210° C. (MeOH); ¹H NMR (300 MHz, d₆-DMSO) δ 2.28 (s, 3H, CH₃), 7.13 (d, 1H, =4.9 Hz, Hₐᵣₒₘ), 7.28-7.41 (m, 5H, Hₐᵣₒₘ), 8.35 (d, 1H, J=4.9 Hz, Hₐᵣₒₘ), 8.44 (s, 1H, Hₐᵣₒₘ), 12.55 (broad s, 1H, NH); MS (SI) m/z 237.

### b) 3-Acetyl-1-benzenesulfonyl-4-phenyl-1H-pyrrolo[2,3-b]pyridine (2f)

The compound 2f is obtained, according to the procedure described for the preparation of 2a, with a yield of 71% from 1f. M.p.=138-140° C. (CH₂Cl₂/pentane); ¹H NMR (300 MHz, CDCl₃) δ 2.19 (s, 3H, CH₃), 7.24 (d, 1H, J=4.9 Hz, Hₐᵣₒₘ), 7.26-7.31 (m, 2H, Hₐᵣₒₘ), 7.42-7.44 (m, 3H, Hₐᵣₒₘ), 7.55 (t, 2H, J=7.5 Hz, Hₐᵣₒₘ), 7.66 (t, 1H, 7.5 Hz, Hₐᵣₒₘ), 8.29-8.32 (m, 3H, 8.49 (d, 1H, J=4.9 Hz, Hₐᵣₒₘ); MS (SI) m/z 377.

### c) 1-(1-Benzenesulfonyl)-4-phenyl-1H-pyrrolo[2,3-b]pyridin-3-yl)-3,3-dimethylaminopropenone (3f)

The compound 3f is obtained, according to the procedure described for the preparation of 3a, with a yield of 65% from 2f. M.p.=179-181° C. (MeOH); ¹H NMR (300 MHz, CDCl₃) δ 2.59 (broad s, 3H, CH₃), 3.01 (broad s, 3H, CH₃), 4.83 (broad d, 1H, J=12.4 Hz, ═CH), 7.29 (d, 1H, J=4.9 Hz, Hₐᵣₒₘ), 7.30-7.40 (m, 6H, ═CH+Hₐᵣₒₘ), 7.59 (t, 2H, J=7.6 Hz, Hₐᵣₒₘ), 7.69 (t, 1H, J=7.6 Hz, Hₐᵣₒₘ), 8.12 (broad s, 1H, Hₐᵣₒₘ), 8.23 (d, 2H, J=7.6 Hz, Hₐᵣₒₘ), 8.41 (d, 1H, J=4.9 Hz, Hₐᵣₒₘ); MS (SI) m/z 432 (M+H⁺).

### d) 3-[(2-Amino)pyrimidin-4-yl]-4-phenyl-1H-pyrrolo[2,3-b]pyridine (4f): meriolin 23

The compound 4f is obtained, according to the procedure described for the preparation of 4a, with a yield of 78% from 3f. M.p.>210° C. (MeOH); ¹H NMR (300 MHz, d₆-DMSO) δ 5.76 (d, 1H, J=5.3 Hz, Hₐᵣₒₘ), 6.03 (broad s, 2H, NH₂), 7.12 (d, 1H, J=4.9 Hz, Hₐᵣₒₘ), 7.25-7.35 (m, 5H, Hₐᵣₒₘ), 7.65 (d, 1H, J=5.3 Hz, Hₐᵣₒₘ), 7.94 (s, 1H, Hₐᵣₒₘ), 8.34 (d, 1H, J=4.9 Hz, Hₐᵣₒₘ), 12.30 (broad s, 1H, NH); MS (SI) m/z 288.

In the same way, the compounds below were prepared from 7-azaindoles substituted in the 4 and/or 6 positions:
3-[(2-amino)pyrimidin-4-yl]-4-butoxy-1H-pyrrolo[2,3-b]pyridine
3-[(2-amino)pyrimidin-4-yl]-4-pentoxy-1H-pyrrolo[2,3-b]pyridine
3-[(2-amino)pyrimidin-4-yl]-4-(3,3,3-trifluoropropoxy)-1H-pyrrolo[2,3-b]pyridine
3-[(2-amino)pyrimidin-4-yl]-4-(3-fluoropropoxy)-1H-pyrrolo[2,3-b]pyridine
3-[(2-amino)pyrimidin-4-yl]-4-(2,2,2-trifluoroethoxy)-1H-pyrrolo[2,3-b]pyridine
3-[(2-amino)pyrimidin-4-yl]-4-(2-fluoroethoxy)-1H-pyrrolo[2,3-b]pyridine
3-[(2-amino)pyrimidin-4-yl]-4-methylthio-1H-pyrrolo[2,3-b]pyridine
3-[(2-amino)pyrimidin-4-yl]-4-ethylthio-1H-pyrrolo[2,3-b]pyridine
3-[(2-amino)pyrimidin-4-yl]-4-propylthio-1H-pyrrolo[2,3-b]pyridine
3-[(2-amino)pyrimidin-4-yl]-4-benzylthio-1H-pyrrolo[2,3-b]pyridine
3-[(2-amino)pyrimidin-4-yl]-4-nitro-1H-pyrrolo[2,3-b]pyridine
3-[(2-amino)pyrimidin-4-yl]-4-methyl-1H-pyrrolo[2,3-b]pyridine
3-[(2-amino)pyrimidin-4-yl]-4-ethyl-1H-pyrrolo[2,3-b]pyridine
3-[(2-amino)pyrimidin-4-yl]-4-propyl-1H-pyrrolo[2,3-b]pyridine
3-[(2-amino)pyrimidin-4-yl]-4-butyl-6-acetylamino-1H-pyrrolo[2,3-b]pyridine
3-[(2-amino)pyrimidin-4-yl]-4-pentyl-6-phenylamino-1H-pyrrolo[2,3-b]pyridine
3-[(2-amino)pyrimidin-4-yl]-4-pentyl-6-benzylamino-1H-pyrrolo[2,3-b]pyridine
3-[(2-amino)pyrimidin-4-yl]-4-pentyl-6-benzyloxy-1H-pyrrolo[2,3-b]pyridine
3-[(2-amino)pyrimidin-4-yl]-4-cyano-1H-pyrrolo[2,3-b]pyridine
methyl 3-[(2-amino)pyrimidin-4-yl]-1H-pyrrolo[2,3-b]pyridine-4-carboxylate
4-amino-3-[(2-amino)pyrimidin-4-yl]-1H-pyrrolo[2,3-b]pyridine
3-[(2-amino)pyrimidin-4-yl]-4-dimethylamino-1H-pyrrolo[2,3-b]pyridine
3-[(2-amino)pyrimidin-4-yl]-4-propylamino-1H-pyrrolo[2,3-b]pyridine
3-[(2-amino)pyrimidin-4-yl]-4-benzylamino-1H-pyrrolo[2,3-b]pyridine
3-[(2-amino)pyrimidin-4-yl]-4-trifluoromethyl-1H-pyrrolo[2,3-b]pyridine
3-[(2-amino)pyrimidin-4-yl]-4-methylsulfonyl-1H-1-pyrrolo[2,3-b]pyridine
3-[(2-amino)pyrimidin-4-yl]-4-bromo-1H-pyrrolo[2,3-b]pyridine
3-[(2-amino)pyrimidin-4-yl]-4-fluoro-1H-pyrrolo[2,3-b]pyridine
3-[(2-amino)pyrimidin-4-yl]-4-propoxy-6-amino-1H-pyrrolo[2,3-b]pyridine
3-[(2-amino)pyrimidin-4-yl]-4-(4-methoxyphenyl)-1H-pyrrolo[2,3-b]pyridine
3-[(2-amino)pyrimidin-4-yl]-4-(4-chlorophenyl)-1H-pyrrolo[2,3-b]pyridine
3-[(2-amino)pyrimidin-4-yl]-4-(4-fluorophenyl)-1H-pyrrolo[2,3-b]pyridine
3-[(2-amino)pyrimidin-4-yl]-4-(3-fluorophenyl)-1H-pyrrolo[2,3-b]pyridine
3-[(2-amino)pyrimidin-4-yl]-4-(3-chlorophenyl)-1H-pyrrolo[2,3-b]pyridine
3-[(2-amino)pyrimidin-4-yl]-4-(3-methoxyphenyl)-1H-pyrrolo[2,3-b]pyridine
3-[(2-amino)pyrimidin-4-yl]-4-methoxy-6-acetylamino-1H-pyrrolo[2,3-b]pyridine
3-[(2-amino)pyrimidin-4-yl]-4-methoxy-6-phenylamino-1H-pyrrolo[2,3-b]pyridine
3-[(2-amino)pyrimidin-4-yl]-4-ethoxy-6-acetylamino-1H-pyrrolo[2,3-b]pyridine
3-[(2-amino)pyrimidin-4-yl]-4-ethoxy-6-phenylamino-1H-pyrrolo[2,3-b]pyridine
3-[(2-amino)pyrimidin-4-yl]-4-propoxy-6-acetylamino-1H-pyrrolo[2,3-b]pyridine
3-[(2-amino)pyrimidin-4-yl]-4-propoxy-6-phenylamino-1H-pyrrolo[2,3-b]pyridine
3-[(2-amino)pyrimidin-4-yl]-4-methoxy-6-benzylamino-1H-pyrrolo[2,3-b]pyridine
3-[(2-amino)pyrimidin-4-yl]-4-ethoxy-6-benzylamino-1H-pyrrolo[2,3-b]pyridine
3-[(2-amino)pyrimidin-4-yl]-4-propoxy-6-benzylamino-1H-pyrrolo[2,3-b]pyridine
3-[(2-amino)pyrimidin-4-yl]-4-methoxy-6-benzyloxy-1H-pyrrolo[2,3-b]pyridine
3-[(2-amino)pyrimidin-4-yl]-4-ethoxy-6-benzyloxy-1H-pyrrolo[2,3-b]pyridine
3-[(2-amino)pyrimidin-4-yl]-4-propoxy-6-benzyloxy-1H-pyrrolo[2,3-b]pyridine
3-[(2-amino)pyrimidin-4-yl]-4-methoxy-6-methylamino-1H-pyrrolo[2,3-b]pyridine
3-[(2-amino)pyrimidin-4-yl]-4-ethoxy-6-methylamino-1H-pyrrolo[2,3-b]pyridine
3-[(2-amino)pyrimidin-4-yl]-4-propoxy-6-methylamino-1H-pyrrolo[2,3-b]pyridine
3-[(2-amino)pyrimidin-4-yl]-4-methoxy-6-cyclohexylamino-1H-pyrrolo[2,3-b]pyridine
3-[(2-amino)pyrimidin-4-yl]-4-ethoxy-6-cyclohexylamino-1H-pyrrolo[2,3-b]pyridine
3-[(2-amino)pyrimidin-4-yl]-4-propoxy-6-cyclohexylamino-1H-pyrrolo[2,3-b]pyridine
3-[(2-amino)pyrimidin-4-yl]-4-methoxy-6-(pyridin-4-ylmethyl)amino-1H-pyrrolo[2,3-b]pyridine
3-[(2-amino)pyrimidin-4-yl]-4-ethoxy-6-(pyridin-4-ylmethyl)amino-1H-pyrrolo[2,3-b]pyridine
3-[(2-amino)pyrimidin-4-yl]-4-propoxy-6-(pyridin-4-ylmethyl)amino-1H-pyrrolo[2,3-b]pyridine
3-[(2-amino)pyrimidin-4-yl]-4-ethoxy-6-[4-(phenyl)benzyloxy]-1H-pyrrolo[2,3-b]pyridine
3-[(2-amino)pyrimidin-4-yl]-4-propoxy-6-[4-(phenyl)benzyloxy]-1H-pyrrolo[2,3-b]pyridine
3-[(2-amino)pyrimidin-4-yl]-4-propoxy-6-[4-(2-pyridinyl)benzyloxy]-1H-pyrrolo[2,3-b]pyridine
3-[(2-amino)pyrimidin-4-yl]-4-propoxy-6-[4-(2-thienyl)benzyloxy]-1H-pyrrolo[2,3-b]pyridine
3-[(2-amino)pyrimidin-4-yl]-4-propoxy-6-[4-(2-thienyl)benzylamino]-1H-pyrrolo[2,3-b]pyridine

### EXAMPLE 8

### Synthesis of 3-[(2-amino)pyrimidin-4-yl]-4-(1-methylethoxy)-1H-pyrrolo[2,3-b]pyridine (4g): meriolin 6

### a) Iodo-4-(1-methylethoxy)-1H-pyrrolo[2,3-b]pyridine (5g)

A solution of iodine (74 mg, 10.29 mmol) in anhydrous DMF (0.5 ml) is added dropwise, at ambient temperature and under an inert atmosphere, to a solution of 4-(1-methylethoxy)-1H-pyrrolo[2,3-b]pyridine (50 mg, 0.28 mmol) and KOH (88 mg, 1.56 mmol) in DMF (0.5 ml). The solution is stirred at ambient temperature for 2.5 h. The solvent is evaporated and then the residue is taken up in H₂O. The suspension obtained is filtered in order to give the compound 5g (70 mg, 82%). M.p. 181-183° C. (MeOH); ¹H NMR (300 MHz, CDCl₃) δ 1.49 (d, 6H, J=6.0 Hz, CH₃), 4.80 (hept, 1H, J=6.0 Hz, CH), 6.55 (d, 1H, J=5.8 Hz, Hₐᵣₒₘ), 7.26 (s, 1H, Hₐᵣₒₘ), 8.14 (d, 1H, J=5.8 Hz, Hₐᵣₒₘ); MS (SI) m/z 303 (M+H⁺).

### b) 1-Benzenesulfonyl-3-iodo-4-(1-methylethoxy)-1H-pyrrolo[2,3-b]pyridine (60

The compound 6g is obtained, according to the procedure described for the preparation of 2a, with a yield of 68% from 5g. M.p.=151-153° C. (CH₂Cl₂/pentane); ¹H NMR (300 MHz, CDCl₃) δ 1.42 (d, 6H, J=6.2 Hz, CH₃), 4.71 (hept, 1H, J=6.2 Hz, CH), 6.59 (d, 1H, J=5.7 Hz, Hₐᵣₒₘ), 7.48 (broad t, 2H, J=7.9 Hz, Hₐᵣₒₘ), 7.58 (broad t, 1H, J=7.9 Hz, Hₐᵣₒₘ), 7.69 (s, 1H, Hₐᵣₒₘ), 8.17 (broad d, 2H, J=7.9 Hz, Hₐᵣₒₘ), 8.27 (d, 1H, J=5.7 Hz, Hₐᵣₒₘ); MS (SI) m/z 443 (M+H⁺).

### e) 3-Acetyl-1-(1-benzenesulfonyl)-4-(1-methylethoxy)-1-pyrrolo[2,3-b]pyridine (2g)

Tri-n-butyl(1-ethoxyvinyl)stannane (77 μl, 0.23 mmol) is added to a solution of 6g (67 mg, 0.15 mmol), freshly prepared Pd(PPh₃)₄ (18 mg, 0.015 mmol) and LiCl (16 mg, 0.38 mmol) in anhydrous DMF (3 ml). The solution is stirred at 80° C. for 18h. After cooling, a 5% HCl solution (10 ml) is added and the final solution is stirred at ambient temperature for 20 min. The solvents are evaporated under reduced pressure. The residue obtained is taken up in a mixture of a saturated Na₂CO₃ solution and AcOEt (pH=7-8) and then the two phases are separated. The organic phase collected is washed with a KF solution, dried over MgSO₄ and then evaporated. The solid obtained is purified with a chromatography column (eluent: petroleum ether/AcOEt 7:3) to give the compound 2g (48 mg, 89%). M.p.=118-120° C. (CH₂Cl₂/petroleum ether); ¹H NMR (300 MHz, CDCl₃) δ 1.43 (d, 6H, J=6.0 Hz, CH₃), 2.65 (s, 3H, CH₃), 4.78 (hept, 1H, J=6.0 Hz, CH), 6.69 (d, 1H, J=6.0 Hz, Hₐᵣₒₘ), 7.51 (broad t, 2H, J=7.9 Hz, Hₐᵣₒₘ), 7.61 (broad t, 1H, J=7.9 Hz, Hₐᵣₒₘ), 8.17 (s, 1H, Hₐᵣₒₘ), 8.23 (broad d, 2H, J=7.9 Hz, Hₐᵣₒₘ), 8.29 (d, 1H, J=6.0 Hz, Hₐᵣₒₘ); MS (SI) m/z 359 (M+H⁺).

### d) 1-(1-Benzenesulfonyl)-4-(1-methylethoxy)-1H-pyrrolo[2,3-b]pyridin-3-yl)-3,3-dimethylaminopropenone (3g)

The compound 3g is obtained, according to the procedure described for the preparation of 3a, with a yield of 69% from 2g. M.p.=150-152° C. (MeOH); ¹H NMR (300 MHz, CDCl₃) δ 1.37 (d, 6H, J=6.0 Hz, CH₃), 2.95 (broad s, 3H, CH₃), 3.14 (broad s, 3H, CH₃), 4.72 (hept, 1H, J=6.0 Hz, CH), 5.68 (broad d, 1H, J=12.5 Hz, CH), 6.65 (d, 1H, J=5.7 Hz, Hₐᵣₒₘ), 7.47 (t, 2H, J=7.5 Hz, Hₐᵣₒₘ), 7.57 (t, 1H, J=7.5 Hz, Hₐᵣₒₘ), 7.73-7.79 (m, 1H, ═CH), 7.98 (s, 1H, Hₐᵣₒₘ), 8.20 (d, 2H, J=7.5 Hz, Hₐᵣₒₘ), 8.28 (d, 1H, J=5.7 Hz, Hₐᵣₒₘ); MS (SI) m/z 414 (M+H⁺).

### e) 3-[(2-amino)pyrimidin-4-yl]-4-(1-methylethoxy)-1H-pyrrolo[2,3-b]pyridine (4g)

The compound 4g is obtained, according to the procedure described for the preparation of 4a, with a yield of 60% from 3g. M.p.>210° C. (MeOH); ¹H NMR (300 MHz, d₆-DMSO) δ 1.39 (d, 6H, J=6.0 Hz, CH₃), 4.91 (hept, 1H, J=6.0 Hz, CH), 6.30 (broad s, 2H, NH₂), 6.77 (d, 1H, J=5.6 Hz, Hₐᵣₒₘ), 7.34 (d, 1H, J=5.3 Hz, Hₐᵣₒₘ), 7.87 (s, 1H, Hₐᵣₒₘ), 8.11 (d, 1H, J=5.6 Hz, Hₐᵣₒₘ), 8.17 (d, 1H, J=5.3 Hz, Hₐᵣₒₘ), 12.03 (broad s, 1H, NH); MS (SI) m/z 270 (M+H⁺).

### EXAMPLE 9

### Synthesis of 3-[(2-amino)pyrimidin-4-yl]-4-(benzyloxy)-1H-pyrrolo[2,3-b]pyridine (4h): meriolin 15

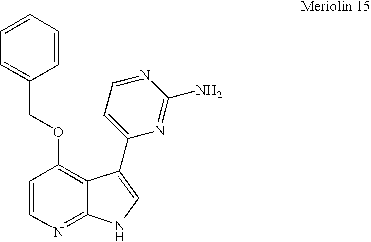

### a) 4-(Benzyloxy)-3-indo-1H-pyrrolo[2,3-b]pyridine (5h)

The compound 5h is obtained, according to the procedure described for the preparation of 5g, with a yield of 88% from 4-(benzyloxy)-1H-pyrrolo[2,3-b]pyridine. M.p.=180-182° C. (CH₂Cl₂); ¹H NMR (300 MHz, d₆-DMSO) δ 5.33 (s, 2H, CH₂), 6.77 (d, 1H, J=5.3 Hz, Hₐᵣₒₘ), 7.30-7.44 (m, 3H, Hₐᵣₒₘ), 7.48 (s, 1H, Hₐᵣₒₘ), 7.62 (broad d, 2H, J=7.4 Hz, Hₐᵣₒₘ), 8.11 (d, 1H, J=5.3 Hz, Hₐᵣₒₘ), 11.95 (broad s, 1H, NH); MS (SI) m/z 351 (M+H⁺).

### b) 1-Benzenesulfonyl-3-iodo-4-(benzyloxy)-1H-pyrrolo[2,3-b]pyridine (6h)

The compound 6h is obtained, according to the procedure described for the preparation of 5g, with a yield of 77% from 5h. M.p.=177-179° C. (CH₂Cl₂/pentane); ¹H NMR (300 MHz, CDCl₃) δ 5.23 (s, 2H, CH₂), 6.69 (d, 1H, J=5.7 Hz, Hₐᵣₒₘ), 7.31-7.61 (m, 8H, Hₐᵣₒₘ), 7.73 (s, 1H, Hₐᵣₒₘ), 8.18 (broad d, 2H, J=7.9 Hz, Hₐᵣₒₘ), 8.29 (d, 1H, J=5.7 Hz, Hₐᵣₒₘ); MS (SI) m/z 491 (M+H⁺).

### c) 3-Acetyl-1-(1-benzenesulfonyl)-4-(benzyloxy)-1H-pyrrolo[2,3-b]pyridine (2h)

The compound 2g is obtained, according to the procedure described for the preparation of 2g, with a yield of 86% from 6g. M.p.=164-166° C. (CH₂Cl₂/EP); ¹H NMR (300 MHz, CDCl₃) δ 2.53 (s, 3H, CH₃), 5.23 (s, 2H, CH₂), 6.78 (d, 1H, J=5.6 Hz, Hₐᵣₒₘ), 7.28-7.61 (m, 8H, Hₐᵣₒₘ), 8.20 (s, 1H, Hₐᵣₒₘ), 8.23 (broad d, 2H, J=7.5 Hz, Hₐᵣₒₘ), 8.29 (d, 1H, J=5.6 Hz, Hₐᵣₒₘ); MS (SI) m/z 407 (M+H⁺).

### d) 1-(1-Benzenesulfonyl-4-(benzyloxy)-1H-pyrrolo[2,3-b]pyridin-3-yl)-3-dimethylaminopropenone (3h)

The compound 3h is obtained, according to the procedure described for the preparation of 3a, with a yield of 66% from 2h. M.p.=190-192° C. (MeOH); ¹H NMR (300 MHz, CD₃OD) δ 2.56 (broad s, 3H, CH₃), 3.06 (broad s, 3H, CH₃), 5.27 (s, 2H, CH₂), 5.61 (broad d, 1H, J=11.5 Hz, ═CH), 7.00 (d, 1H, J=5.8 Hz, Hₐᵣₒₘ), 7.34-7.69 (m, 9H, CH+Hₐᵣₒₘ), 7.99 (s, 1H, Hₐᵣₒₘ), 8.17 (d, 2H, J=7.4 Hz, Hₐᵣₒₘ), 8.24 (d, 1H, J=5.8 Hz, Hₐᵣₒₘ); MS (SI) m/z 462 (M+H⁺).

### e) 3-[(2-amino)pyrimidin-4-yl]-4-(benzyloxy)-1H-pyrrolo[2,3-b]pyridine (4h): meriolin 15

The compound 4h is obtained, according to the procedure described for the preparation of 4a, with a yield of 90% from 3h. M.p.>210° C. (MeOH); ¹H NMR (300 MHz, d₆-DMSO) δ 5.34 (s, 2H, CH₂), 6.29 (broad s, 2H, NH₂), 6.90 (d, 1H, J=5.5 Hz, Hₐᵣₒₘ), 7.23 (d, 1H, J=5.3 Hz, Hₐᵣₒₘ), 7.37-7.45 (m, 3H, Hₐᵣₒₘ), 7.52-7.54 (m, 2H, Hₐᵣₒₘ), 7.85 (d, 1H, J=5.3 Hz, Hₐᵣₒₘ), 7.90 (s, 1H, Hₐᵣₒₘ), 8.16 (d, 1H, J=5.5 Hz, Hₐᵣₒₘ), 12.12 (broad s, 1H, NH); MS (SI) m/z 318 (M++H⁺).

### EXAMPLE 10

### Synthesis of 3-[(2-amino)pyrimidin-4-yl]-4-(cyclohexyloxy)-1H-pyrrolo[2,3-b]pyridine (4i): meriolin 17

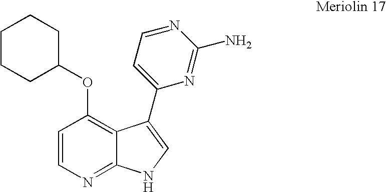

### a) 4-(Cyclohexyloxy)-3-iodo-1H-pyrrolo[2,3-b]pyridine (5i)

The compound 5i is obtained, according to the procedure described for the preparation of 5g, with a yield of 82% from 4-(cyclohexyloxy)-1H-pyrrolo[2,3-b]pyridine. M.p.=199-201° C. (H₂O); ¹H NMR (300 MHz, CDCl₃) δ 1.40-1.60 (m, 5H, CH₂), 1.82-2.00 (m, 5H, CH₂), 4.67 (broad s, 1H, CH), 6.62 (d, 1H, J=6.1 Hz, Hₐᵣₒₘ), 7.30 (s, 1H, Hₐᵣₒₘ), 8.12 (d, 1H, J=6.1 Hz, Hₐᵣₒₘ); MS (SI) m/z 343 (M+H⁺).

### b) 1-Benzenesulfonyl-3-iodo-4-(cyclohexyloxy)-1H-pyrrolo[2,3-b]pyridine (6i)

The compound 6i is obtained, according to the procedure described for the preparation of 5g, with a yield of 85% from 5i. M.p.=112-114° C. (CH₂Cl₂/pentane); ¹H NMR (300 MHz, CDCl₃) δ 1.40-1.60 (m, 5H, CH₂), 1.70-2.00 (m, 5H, CH₂), 4.52 (broad s, 1H, CH), 6.60 (d, 1H, J=5.7 Hz, Hₐᵣₒₘ), 7.48 (t, 2H, J=7.2 Hz, Hₐᵣₒₘ), 7.58 (t, 1H, J=7.7 Hz, Hₐᵣₒₘ), 7.69 (s, 1H, Hₐᵣₒₘ), 8.18 (broad d, 2H, J=8.0 Hz, Hₐᵣₒₘ), 8.26 (d, 1H, J=5.7 Hz, Hₐᵣₒₘ); MS (SI) m/z 483 (M+H⁺).

### c) 3-Acetyl-1-(1-benzenesulfonyl)-4-(cyclohexyloxy)-1H-pyrrolo[2,3-b]pyridine (2i)

The compound 2i is obtained, according to the procedure described for the preparation of 2g, with a yield of 73% from 6i. M.p.=107-109° C. (CH₂Cl₂/EP); ¹H NMR (300 MHz, CDCl₃) δ 1.29-1.44 (m, 3H, CH₂), 1.50-1.70 (m, 3H, CH₂), 1.80-1.90 (m, 2H, CH₂), 2.00-2.10 (m, 2H, CH₂), 2.64 (s, 3H, CH₃), 4.44-4.52 (m, 1H, CH), 6.70 (d, 1H, J=5.7 Hz, Hₐᵣₒₘ), 7.49 (t, 2H, J=7.4 Hz, Hₐᵣₒₘ), 7.59 (t, 1H, J=7.2 Hz, Hₐᵣₒₘ), 8.15 (s, 1H, Hₐᵣₒₘ), 8.22 (broad d, 2H, J=7.7 Hz, Hₐᵣₒₘ), 8.27 (d, 1H, J=5.7 Hz, Hₐᵣₒₘ); MS (SI) m/z 399 (M+H⁺).

### d) 1-(1-Benzenesulfonyl-4-(cyclohexyloxy)-1H-pyrrolo[2,3-b]pyridin-3-yl)-3-dimethylaminopropenone (3i)

The compound 3i is obtained, according to the procedure described for the preparation of 3a, with a yield of 72% from 2i. M.p.=95-97° C. (MeOH); ¹H NMR (300 MHz, CDCl₃) δ 1.29-1.90 (m, 1H, CH₂), 2.95 (broad s, 3H, CH₃), 3.04 (broad s, 3H, CH₃), 4.41-4.48 (m, 1H, CH), 5.53 (d, 1H, J=12.6 Hz, ═CH), 6.62 (d, 1H, J=5.7 Hz, Hₐᵣₒₘ), 7.40-7.55 (m, 4H, ═CH+Hₐᵣₒₘ), 7.85 (s, 1H, Hₐᵣₒₘ), 8.16 (d, 2H, J=7.9 Hz, Hₐᵣₒₘ), 8.22 (d, 1H, J=5.7 Hz, Hₐᵣₒₘ); MS (SI) m/z 454 (M+H⁺).

### e) 3-[(2-amino)pyrimidin-4-yl]-4-(cyclohexyloxy)-1H-pyrrolo[2,3-b]pyridine (4i): meriolin 17

The compound 4i is obtained, according to the procedure described for the preparation of 4a, with a yield of 65% from 3i. M.p.>210° C. (MeOH); ¹H NMR (300 MHz, d₆-DMSO) δ 1.28-1.63 (m, 6H, CH₂), 1.69 (broad s, 2H, CH₂), 2.01 (broad s, 2H, CH₂), 4.64-4.70 (m, 1H, CH), 6.31 (broad s, 2H, NH₂), 6.79 (d, 1H, J=5.5 Hz, Hₐᵣₒₘ), 7.31 (d, J=5.3 Hz, Hₐᵣₒₘ), 7.86 (d, 1H, J=2.3 Hz, Hₐᵣₒₘ), 8.10 (d, 1H, J=5.5 Hz, Hₐᵣₒₘ), 8.16 (d, 1H, J=5.3 Hz, Hₐᵣₒₘ), 12.04 (broad s, 1H, NH); MS (SI) m/z 310 (M+H⁺).

In the same way, the compounds below were prepared from 7-azaindoles substituted in the 4 position:
3-[(2-amino)pyrimidin-4-yl]-4-(1-ethylpropoxy)-1H-pyrrolo[2,3-b]pyridine
3-[(2-amino)pyrimidin-4-yl]-4-(1-methylethoxy)-1H-pyrrolo[2,3-b]pyridine
3-[(2-amino)pyrimidin-4-yl]-4-(1,1-dimethylethoxy)-1H-pyrrolo[2,3-b]pyridine
3-[(2-amino)pyrimidin-4-yl]-4-(1-methylpropoxy)-1H-pyrrolo[2,3-b]pyridine
3-[(2-amino)pyrimidin-4-yl]-4-(1-cyclopentoxy)-1H-pyrrolo[2,3-b]pyridine
3-[(2-amino)pyrimidin-4-yl]-4-(1-cycloheptyloxy)-1H-pyrrolo[2,3-b]pyridine
3-[(2-amino)pyrimidin-4-yl]-4-(4-methoxybenzyloxy)-1H-pyrrolo[2,3-b]pyridine
3-[(2-amino)pyrimidin-4-yl]-4-(3-methoxybenzyloxy)-1H-pyrrolo[2,3-b]pyridine
3-[(2-amino)pyrimidin-4-yl]-4-(2-methoxybenzyloxy)-1H-pyrrolo[2,3-b]pyridine
3-[(2-amino)pyrimidin-4-yl]-4-(4-chlorobenzyloxy)-1H-pyrrolo[2,3-b]pyridine
-3-[(2-amino)pyrimidin-4-yl]-4-(3-chlorobenzyloxy)-1H-pyrrolo[2,3-b]pyridine
3-[(2-amino)pyrimidin-4-yl]-4-(2-chlorobenzyloxy)-1H-pyrrolo[2,3-b]pyridine
3-[(2-amino)pyrimidin-4-yl]-4-(4-fluorobenzyloxy)-1H-pyrrolo[2,3-b]pyridine
3-[(2-amino)pyrimidin-4-yl]-4-(3-fluorobenzyloxy)-1H-pyrrolo[2,3-b]pyridine
3-[(2-amino)pyrimidin-4-yl]-4-(4-hydroxybenzyloxy)-1H-pyrrolo[2,3-b]pyridine
3-[(2-amino)pyrimidin-4-yl]-4-(3-hydroxybenzyloxy)-1H-pyrrolo[2,3-b]pyridine
3-[(2-amino)pyrimidin-4-yl]-4-(4-methanesulfonylbenzyloxy)-1H-pyrrolo[2,3-b]pyridine
3-[(2-amino)pyrimidin-4-yl]-4-(pyridin-4-ylmethoxy)-1H-pyrrolo[2,3-b]pyridine
3-[(2-amino)pyrimidin-4-yl]-4-(pyridin-3-ylmethoxy)-1H-pyrrolo[2,3-b]pyridine

3-[(2-amino)pyrimidin-4-yl]-4-(pyridin-2-ylmethoxy)-1H-pyrrolo[2,3-b]pyridine
3-[(2-amino)pyrimidin-4-yl]-4-(pyrimidin-5-ylmethoxy)-1H-pyrrolo[2,3-b]pyridine
-3-[(2-amino)pyrimidin-4-yl]-4-(piperidin-4-ylmethoxy)-1H-pyrrolo[2,3-b]pyridine
3-[(2-amino)pyrimidin-4-yl]-4-(piperidin-4-ylmethoxy)-1H-pyrrolo[2,3-b]pyridine
3-[(2-amino)pyrimidin-4-yl]-4-(1-methanesulfonylpipetidin-4-ylmethoxy)-1H-pyrrolo[2,3-b]pyridine
3-[(2-amino)pyrimidin-4-yl]-4-benzyloxy-6-[4-(2-pyridinyl)benzyloxy]-1H-pyrrolo[2,3-b]pyridine
3-[(2-amino)pyrimidin-4-yl]-4-benzyloxy-6-[4-(3-pyridinyl)benzyloxy]-1H-pyrrolo[2,3-b]pyridine
3-[(2-amino)pyrimidin-4-yl]-4-benzyloxy-6-[4-(2-pyridinyl)benzylamino]-1H-pyrrolo[2,3-b]pyridine
3-[(2-amino)pyrimidin-4-yl]-4-benzyloxy-6-[4-(3-pyridinyl)benzylamino]-1H-pyrrolo[2,3-b]pyridine

### EXAMPLE 11

### Synthesis of 4-methoxy-3-(pyrimidin-4-yl)-1H-pyrrolo[2,3-b]pyridine (7): meriolin 14

A solution of 4-methoxy-7-azaindole (100 mg, 0.67 mmol), CuCl (27 mg, 0.27 mmol) and formamide (300 μl) is heated at 170° C. for 18 h. After cooling, H₂O is added to the solution (pH=9) and then this solution is extracted with AcOEt. The organic phase is dried over MgSO₄ and then evaporated. The residue is purified with a chromatography column (eluent: AcOEt/MeOH 9:1) to give 7 (40 mg, 26%). M.p.>210° C. (MeOH); ¹H NMR (300 MHz, CD₃OD+D₂O) δ 4.09 (s, 3H, CH₃), 6.88 (d, 1H, J=5.6 Hz, Hₐᵣₒₘ), 8.12 (s, 1H, Hₐᵣₒₘ), 8.20-8.22 (m, 2H, Hₐᵣₒₘ), 8.65 (d, 1H, J=−5.3 Hz, Hₐᵣₒₘ), 8.99 (s, 1H, Hₐᵣₒₘ); MS (SI) m/z 227 (M+H⁺).

In the same way, the compounds below were prepared from 7-azaindoles substituted in the 4 position:
3-(pyrimidin-4-yl)-4-ethoxy-1H-pyrrolo[2,3-b]pyridine
3-(pyrimidin-4-yl)-4-propoxy-1H-pyrrolo[2,3-b]pyridine
3-(pyrimidin-4-yl)-4-(1-methylethoxy)-1H-pyrrolo[2,3-b]pyridine
3-(pyrimidin-4-yl)-4-nitro-1H-pyrrolo[2,3-b]pyridine
3-(pyrimidin-4-yl)-4-chloro-1H-pyrrolo[2,3-b]pyridine
3-(pyrimidin-4-yl)-4-fluoro-1H-pyrrolo[2,3-b]pyridine
3-(pyrimidin-4-yl)-4-methyl-1H-pyrrolo[2,3-b]pyridine
3-(pyrimidin-4-yl)-4-ethyl-1H-pyrrolo[2,3-b]pyridine
3-(pyrimidin-4-yl)-4-cyano-1H-pyrrolo[2,3-b]pyridine
3-(pyrimidin-4-yl)-4-phenyl-1H-pyrrolo[2,3-b]pyridine
3-(pyrimidin-4-yl)-4-methylamino-1H-pyrrolo[2,3-b]pyridine

### EXAMPLE 12

### Synthesis of 3-[(2-amino)pyrimidin-4-yl]-4-(pent-1-ynyl)-1H-pyrrolo[2,3-b]pyridine (4j): meriolin 18

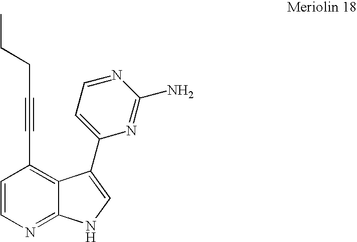

### a) 3-Acetyl-1-(1-benzenesulfonyl)-4-(pent-1-ynyl)-1H-pyrrolo[2,3-b]pyridine (2j)

Triethylamine (250 μl, 1.80 mmol) and pent-1-yne (225 μl, 2.30 mmol) are added to a solution of 2a (150 mg, 0.45 mmol), PdCl₂(PPh₃)₂ (63 mg, 0.09 mmol) and CuI (34 mg, 0.18 mmol) in anhydrous DMF (4.5 ml). The solution is stirred at 50° C. for 72 h in a sealed tube. After cooling, the solution is taken up in a mixture of 1M NH₄OH (50 ml) and CH₂Cl₂ (50 ml). The phases are separated. The aqueous phase is extracted a further time with CH₂Cl₂ (25 ml). The combined organic phases are dried over MgSO₄ and then evaporated. The residue obtained is purified with a chromatography column (eluent: petroleum ether/AcOEt 85:15) to give the compound 2j (110 mg, 67%). M.p.=134-136° C. (CH₂Cl₂/pentane); ¹H NMR (300 MHz, CDCl₃) δ 1.01 (t, 3H, J=7.3 Hz, CH₃), 1.60-1.72 (m, 2H, CH₂), 2.47 (t, 2H, J=7.3 Hz, CH₂), 2.60 (s, 3H, CH₃), 7.25 (d, 1H, J=5.0 Hz, Hₐᵣₒₘ), 7.49 (t, 2H, J=7.4 Hz, Hₐᵣₒₘ), 7.59 (t, 1H, J=7.4 Hz, Hₐᵣₒₘ), 8.20 (d, 2H, J=7.5 Hz, Hₐᵣₒₘ), 8.28 (s, 1H, Hₐᵣₒₘ), 8.31 (d, 1H, J=5.0 Hz, Hₐᵣₒₘ); MS (SI) m/z 367 (M+H⁺).

### b) 1-(1-Benzenesulfonyl-4-(pent-1-ynyl)-1H-pyrrolo[2,3-b]pyridin-3-yl)-3-dimethylaminopropenone (3j)

The compound 3j is obtained, according to the procedure described for the preparation of 3a, with a yield of 66% from 2j. Oil; ¹H NMR (300 MHz, CDCl₃) δ 0.95 (t, 3H, J=7.3 Hz, CH₃), 1.50-1.62 (m, 2H, CH₂), 2.34 (t, 2H, J=7.3 Hz, CH₂), 2.84 (broad s, 3H, CH₃), 3.04 (broad s, 3H, CH₃), 5.45 (d, 1H, J=12.6 Hz, ═CH), 7.15 (d, 1H, J=5.1 Hz, Hₐᵣₒₘ), 7.40-7.55 (m, 4H, ═CH+Hₐᵣₒₘ), 7.94 (s, 1H, Hₐᵣₒₘ), 8.13 (d, 2H, J=7.5 Hz, Hₐᵣₒₘ), 8.28 (d, 1H, J=5.1 Hz, Hₐᵣₒₘ); MS (SI) m/z, 422 (M+H⁺).

### c) 3-[(2-amino)pyrimidin-4-yl]-4-(pent-1-ynyl)-1H-pyrrolo[2,3-b]pyridine (4j): meriolin 18

The compound 4j is obtained, according to the procedure described for the preparation of 4a, with a yield of 50% from 3j. M.p.>210° C. (MeOH); ¹H NMR (300 MHz, d₆-DMSO) δ 0.89 (t, 3H, J=7.3 Hz, CH₃), 1.43-1.55 (m, 2H, CH₂), 2.40 (t, 2H, J=7.3 Hz, CH₂), 6.43 (broad s, 2H, NH₂), 7.01 (d, 1H, J=5.1 Hz, Hₐᵣₒₘ), 7.14 (d, 1H, J=4.9 Hz, Hₐᵣₒₘ), 7.92 (s, 1H, Hₐᵣₒₘ), 8.19 (d, 1H, J=5.5 Hz, Hₐᵣₒₘ), 8.21 (d, 1H, J=5.1 Hz, Hₐᵣₒₘ), 12.29 (broad s, 1H, NH); MS (SI) m/z 278 (M

### EXAMPLE 13

### Synthesis of 3-[(2-amino)pyrimidin-4-yl]-4-(phenyleth-1-ynyl)-1H-pyrrolo[2,3-b]pyridine (4k): meriolin 21

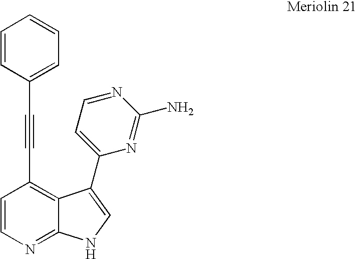

### a) 3-Acetyl-1-(1-benzenesulfonyl)-4-(phenyleth-1-ynyl)-1H-pyrrolo[2,3-b]pyridine (2k)

The compound 2k is obtained, according to the procedure described for the preparation of 2j, with a yield of 33% from 2a. ¹H NMR (300 MHz, CDCl₃) δ 2.66 (s, 3H, CH₃), 7.37-7.42 (m, 4H, Hₐᵣₒₘ), 7.54 (t, 2H, J=7.8 Hz, Hₐᵣₒₘ), 7.62-7.71 (m, 3H, Hₐᵣₒₘ), 8.23-8.27 (m, 2H, Hₐᵣₒₘ), 8.36 (s, 1H, Hₐᵣₒₘ), 8.40 (d, 1H, J=5.1 Hz, Hₐᵣₒₘ); MS (SI) m/z 401 (M+H⁺).

### b) 1-(1-Benzenesulfonyl-4-(phenyleth-1-ynyl)-1H-pyrrolo[2,3-b]pyridin-3-yl)-3-dimethylaminopropenone (3k)

The compound 3k is obtained, according to the procedure described for the preparation of 3a, with a yield of 43% from 2k. M.p. 94-96° C. (MeOH); ¹H NMR (300 MHz, CD₃OD+D₂O) δ 2.81 (broad s, 3H, CH₃), 3.07 (broad s, 3H, CH₃), 5.62 (d, 1H, J=12.4 Hz, ═CH), 7.39-7.42 (m, 4H, Hₐᵣₒₘ), 7.54-7.72 (m, 6H, ═CH+Hₐᵣₒₘ), 8.15 (s, 1H, Hₐᵣₒₘ), 8.20 (d, 2H, J=7.5 Hz, Hₐᵣₒₘ), 8.36 (d, 1H-1, 4.8 Hz, Hₐᵣₒₘ); MS (SI) m/z 456 (M+H⁺).

### c) 3-[(2-amino)pyrimidin-4-yl]-4-(phenyleth-1-ynyl)-1H-pyrrolo-[2,3-b]pyridine (4k): meriolin 21

The compound 4k is obtained, according to the procedure described for the preparation of 4a, with a yield of 73% from 3k. M.p.>210° C. (MeOH); ¹H NMR (300 MHz, CD₃OD+D₂O) δ 7.20 (d, 1H, J=5.3 Hz, Hₐᵣₒₘ), 7.36 (d, 1H, J=4.9 Hz, Hₐᵣₒₘ), 7.38-7.42 (m, 5H, Hₐᵣₒₘ), 7.95 (s, 1H, Hₐᵣₒₘ), 8.18 (d, 1H, J=5.3 Hz, Hₐᵣₒₘ), 8.29 (d, 1H, J=4.9 Hz, Hₐᵣₒₘ); MS (SI) m/z 312 (M+H⁺).

In the same way, the compounds below were prepared from 2a:
3-[(2-amino)pyrimidin-4-yl]-4-(prop-1-ynyl)-1H-pyrrolo[2,3-b]pyridine
3-[(2-amino)pyrimidin-4-yl]-4-(but-1-ynyl)-1H-pyrrolo[2,3-b]pyridine
3-[(2-amino)pyrimidin-4-yl]-4-(hex-1-ynyl)-1H-pyrrolo[2,3-b]pyridine
3-[(2-amino)pyrimidin-4-yl]-4-(4-methylpent-1-ynyl)-1H-pyrrolo[2,3-b]pyridine
3-[(2-amino)pyrimidin-4-yl]-4-(4-cyclohexyleth-1-ynyl)-1H-pyrrolo[2,3-b]pyridine

The compounds 4l and 4m of the invention are synthesized by a catalytic hydrogenation reaction of the alkynes 4j and 4k respectively. This process is represented in scheme 2 below:

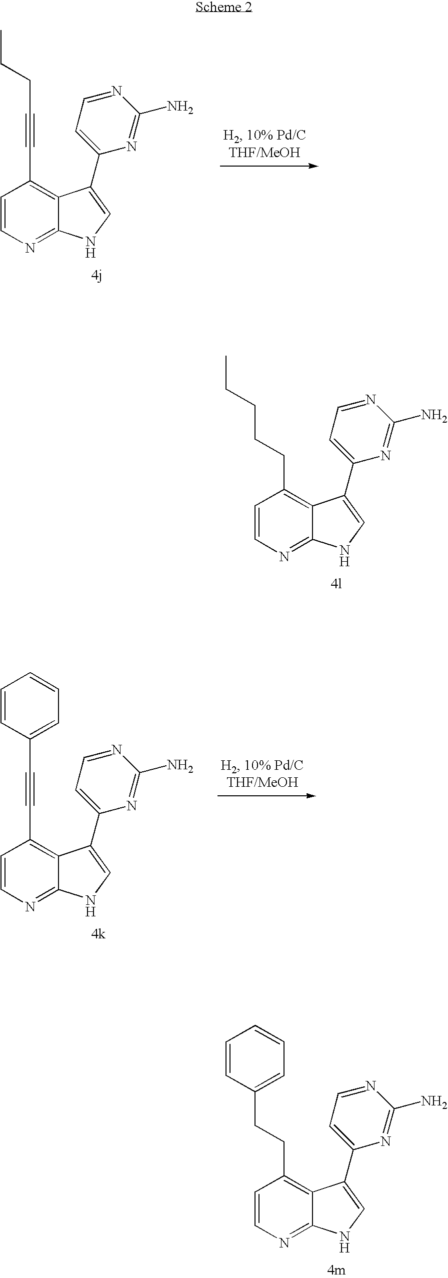

### EXAMPLE 14

### Synthesis of 3-[(2-amino)pyrimidin-4-yl]-4-pentyl-1H-pyrrolo[2,3-b]pyridine (4l): meriolin 19

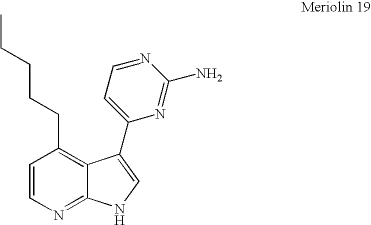

The compound 4j (28 mg, 0.1 mmol) and 10% Pd/C (9 mg) in a THF/MeOH mixture (2 ml; 1:1) are stirred under a pressure of 5 atm of dihydrogen for 24 h. After evaporating the solvents, the residue is purified with a chromatography column (eluent: AcOEt/EtOH 9:1) to give the compound 4l (22 mg, 78%). M.p.>210° C. (MeOH); ¹H NMR (300 MHz, d₆-DMSO) δ 0.69 (t, 3H, J=7.0 Hz, CH₃), 1.09-1.23 (m, 6H, CH₂), 3.23 (t, 2H, J=7.9 Hz, CH₂), 6.48 (broad s, 2H, NH₂), 6.82 (d, 1H, J=5.1 Hz, Hₐᵣₒₘ), 6.94 (d, 1H, J=4.9 Hz, Hₐᵣₒₘ), 7.84 (s, 1H, Hₐᵣₒₘ), 8.14 (d, 1H, J=4.9 Hz, Hₐᵣₒₘ), 8.17 (d, 1H, J=5.1 Hz, Hₐᵣₒₘ), 12.03 (broad s, 1H, NH); MS (SI) m/z 282 (M+H⁺).

### EXAMPLE 15

### Synthesis of 3-[(2-amino)pyrimidin-4-yl]-4-phenethyl-1H-pyrrolo[2,3-b]pyridine (4m): meriolin 22

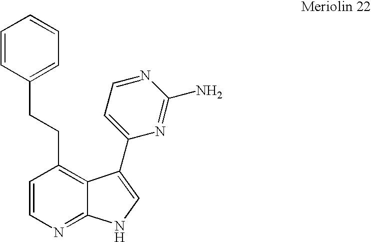

The compound 4l is obtained, according to the procedure described for the preparation of 4l, with a yield of 77% from 4k. M.p.=196-198° C. (MeOH); ¹H NMR (300 MHz, d₆-DMSO) δ 2.56 (broad t, 2H, J=8.1 Hz, CH₂), 3.53 (broad t, 2H, J=8.1 Hz, CH₂), 6.47 (broad s, 1H, NH₂), 6.88 (d, 1H, J=5.1 Hz, Hₐᵣₒₘ), 6.96-6.99 (m, 3H, Hₐᵣₒₘ), 7.11-7.20 (m, 3H, Hₐᵣₒₘ), 7.89 (s, 1H, Hₐᵣₒₘ), 8.15 (d, 1H, J=4.9 Hz, Hₐᵣₒₘ), 8.20 (d, 1H, J=5.1 Hz, Hₐᵣₒₘ); MS (SI) m/z 316 (M+H⁺).

In the same way, the compounds below were prepared:
3-[(2-amino)pyrimidin-4-yl]-4-propyl-1H-pyrrolo[2,3-b]pyridine
3-[(2-amino)pyrimidin-4-yl]-4-butyl-1H-pyrrolo[2,3-b]pyridine
3-[(2-amino)pyrimidin-4-yl]-4-hexyl-1H-pyrrolo[2,3-b]pyridine
3-[(2-amino)pyrimidin-4-yl]-4-(4-methylpentyl)-1H-pyrrolo[2,3-b]pyridine
3-[(2-amino)pyrimidin-4-yl]-4-(4-cyclohexylethyl)-1H-pyrrolo[2,3-b]pyridine

The compounds 4 are prepared according to the same synthetic approach from 1 or 5 but replacing the SO₂Ph protective group with the ethoxymethyl group, as represented in the following scheme 3:

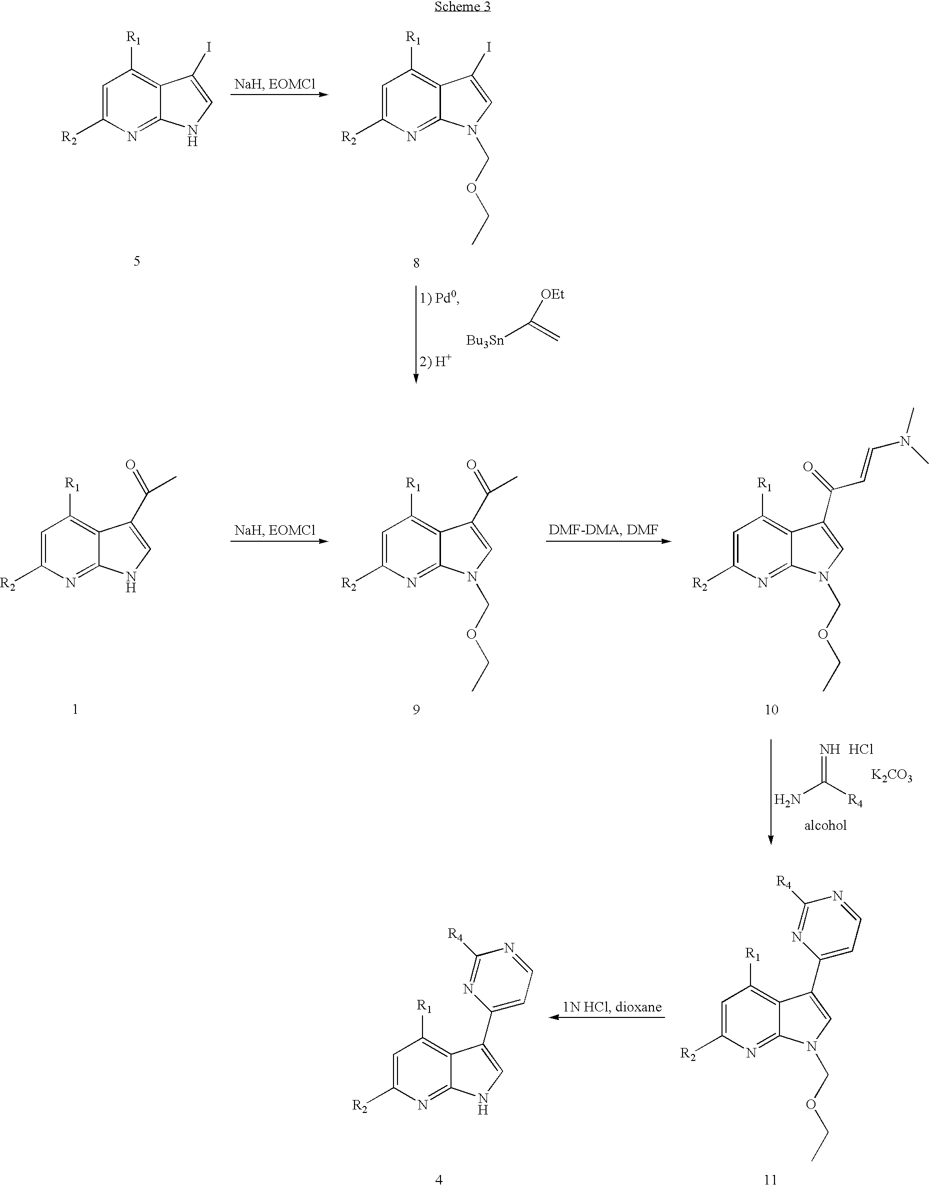

### EXAMPLE 16

### Synthesis of 3-[(2-amino)pyrimidin-4-yl]-6-bromo-4-methoxy-1H-pyrrolo[2,3-b]pyridine (4n)

### a) 3-Acetyl-6-bromo-4-methoxy-1H-pyrrolo[2,3-b]pyridine (1n)

The compound 1n is obtained, according to the procedure described for the preparation of 1a, with a yield of 91% from 6-bromo-4-methoxy-7-azaindole, M.p.>210° C. (MeOH); ¹H NMR (300 MHz, d₅-DMSO) δ 2.50 (s, 3H, CH₃), 3.95 (s, 3H, CH₃), 6.96 (s, 1H, Hₐᵣₒₘ), 8.14 (s, 1H, Hₐᵣₒₘ), 12.55 (broad s, 1H, NH); MS (SI) m/z 271 (⁸¹Br, M+H⁺), 269 (⁷⁹Br, M+H⁺).

### b) 3-Acetyl-6-bromo-1-(1-ethoxymethyl)-4-methoxy-1H-pyrrolo[2,3-b]pyridine (9n)

Sodium hydride (26 mg, 0.64 mmol, 60% in oil) is added in small portions to a solution of 1n (80 mg, 0.30 mmol) in anhydrous DMF (3 ml) at 0° C. The solution is stirred at 0° C. for 45 min and then ethoxymethyl chloride (42 μl, 0.50 mmol) is added to the reaction mixture. The final solution is stirred at ambient temperature for 4 h. The addition of H₂O is carried out at 0° C. and then the solvents are removed under reduced pressure. The residue obtained is taken up in a mixture of H₂O and AcOEt and then the two phases are separated. The organic phase collected is dried over MgSO₄ and then evaporated. The solid obtained is purified with a chromatography column (eluent: petroleum ether/AcOEt 7:3) to give the compound 9n (57 mg, 58%). M.p.=114-116° C. (CH₂Cl₂/pentane); ¹H NMR (300 MHz, CDCl₃) δ 1.18 (t, 3H, J=7.2 Hz, CH₃), 2.62 (s, 3H, CH₃), 3.53 (q, 2H, J=7.2 Hz, CH₂), 4.03 (s, 3H, CH₃), 5.64 (s, 2H, CH₂), 6.84 (s, 1H, Hₐᵣₒₘ), 7.86 (s, 1H, Hₐᵣₒₘ); MS (SI) adz 329 (⁸¹Br, M+H⁺), 327 (⁷⁹Br, M+H⁺).

### c) 6-Bromo-1-(1-ethoxymethyl)-4-methoxy-1H-pyrrolo[2,3-b]pyridin-3-yl)-3,3-dimethylaminopropenone (10n)

The compound 10n is obtained, according to the procedure described for the preparation of 3a, with a yield of 76% from 9n. M.p.=177-179° C. (MeOH); ¹H NMR (300 MHz, CDCl₃) δ 1.11 (t, 3H, J=7.2 Hz, CH₃), 2.90-3.10 (m, 6H, 2 CH₃), 3.46 (q, 2H, J=7.2 Hz, CH₂), 3.96 (s, 3H, CH₃), 5.58 (s, 2H, CH₂), 5.83 (d, 1H, J=12.6 Hz, ═CH), 6.74 (s, 1H, Hₐᵣₒₘ), 7.69 (d, 1H, J=12.4 Hz, ═CH), 7.73 (s, 1H, Hₐᵣₒₘ); MS (SI) m/z 384 (⁸¹Br, M+H⁺), 382 (⁷⁹Br, M+H⁺).

### d) 3-[(2-amino)pyrimidin-4-yl]-6-bromo-1-(1-ethoxymethyl)-4-methoxy-1H-pyrrolo[2,3-b]pyridine (11n)

The compound 11n is obtained, according to the procedure described for the preparation of 4a, with a yield of 95% from 10n. M.p.>210° C. (MeOH); ¹H NMR (300 MHz, CD₃OD+D₂O) δ 1.15 (t, 3H, J=7.2 Hz, CH₃), 3.54 (q, 2H, J=7.2 Hz, CH₂), 4.04 (s, 3H, CH₃), 5.67 (s, 2H, CH₂), 7.00 (s, 1H, Hₐᵣₒₘ), 7.32 (d, 1H, J=5.5 Hz, Hₐᵣₒₘ), 8.04 (s, 1H, Hₐᵣₒₘ) 8.20 (d, 1H, J=5.5 Hz, Hₐᵣₒₘ); MS (SI) m/z 380 (⁸¹Br, M++⁺), 378 (⁷⁹Br, M+H⁺).

### e) 3-[(2-amino)pyrimidin-4-yl]-6-bromo-4-methoxy-1H-pyrrolo[2,3-b]pyridine (4n)

A solution of 11n (50 mg, 0.13 mmol) and 1N HCl (1 ml) in 1,4-dioxane (2 ml) is stirred at 70° C. for 30 min. After cooling, the solution is neutralized by addition of a saturated NaHCO₃ solution and is then extracted with CH₂Cl₂ (2×5 ml). The organic phase is dried over MgSO₄ and then evaporated. The crude solid is recrystallized in MeOH to give 4n (30 mg, 70%). M.p.>210° C. (MeOH); ¹H NMR (300 MHz, CD₃OD+D₂O) δ 4.04 (s, 3H, CH₃), 6.95 (s, 1H, Hₐᵣₒₘ), 7.32 (d, 1H, J=5.5 Hz, Hₐᵣₒₘ), 7.97 (s, 1H, Hₐᵣₒₘ), 8.20 (d, 1H, J=5.5 Hz, Hₐᵣₒₘ); MS (SI) m/z 322 (⁸¹Br, M+Hⁱ), 320 (⁷⁹Br, M+H⁺).

The compounds below were prepared in an identical fashion from 6-bromo-4-ethyloxy-1H-pyrrolo[2,3-b]pyridine and 6-bromo-4-propyloxy-1H-pyrrolo[2,3-b]pyridine:
3-[(2-amino)pyrimidin-4-yl]-6-bromo-4-ethoxy-1H-pyrrolo[2,3-b]pyridine
3-[(2-amino)pyrimidin-4-yl]-6-bromo-4-propoxy-1H-pyrrolo[2,3-b]pyridine
3-[(2-amino)pyrimidin-4-yl]-6-chloro-4-ethoxy-1H-pyrrolo[2,3-b]pyridine
3-[(2-amino)pyrimidin-4-yl]-6-chloro-4-propoxy-1H-pyrrolo[2,3-b]pyridine
3-[(2-amino)pyrimidin-4-yl]-6-fluoro-4-ethoxy-1H-pyrrolo[2,3-b]pyridine
3-[(2-amino)pyrimidin-4-yl]-6-fluoro-4-propoxy-1H-pyrrolo[2,3-b]pyridine

Another process for the synthesis of the meriolins of the invention involves the N-alkylation (NaH, EX) or N-arylation (ArX, Cu₂O, K₂CO₃) of the compounds 1, which results in the products 12 being obtained. The enaninones 13 are prepared by treatment of 12 in the presence of DMF-DMA (Tetrahedron, 2001, 57, 2355-2363). The final compounds 14 are obtained by heating 13 in the presence of guanidinium hydrochloride or derivatives.

This process is represented in scheme 4 below.

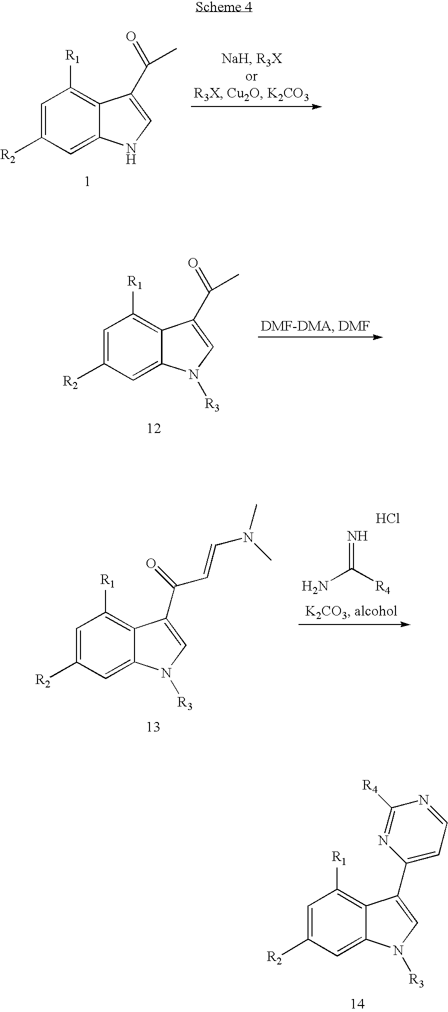

### EXAMPLE 17

### Synthesis of 3-[(2-amino)pyrimidin-4-yl]-4-methoxy-1-methyl-1H-pyrrolo[2,3-b]pyridine (14b): meriolin 9

### a) 3-Acetyl-4-methoxy-1-methyl-1H-pyrrolo[2,3-b]pyridine (12b)

The solution of 1b (80 mg, 0.42 mmol), dimethyl sulfate (0.07 ml, 0.74 mmol) and K₂CO₃ (82 mg, 0.59 mmol) in anhydrous acetone (15 ml) is heated at reflux for 5.5 h under an inert atmosphere. After cooling, the solvent is evaporated. The residue obtained is taken up in a mixture of H₂O and AcOEt (30 ml, 1:1) and then the two phases are separated. The organic phase collected is dried over MgSO₄ and then evaporated. The solid obtained is purified with a chromatography column (eluent: AcOEt) to give the compound 12b (70 mg, 81%). M.p.=79-81° C. (CH₂Cl₂/hexane); ¹H NMR (300 MHz, CDCl₃) δ 2.61 (s, 3H, CH₃), 3.85 (s, 3H, CH₃), 3.99 (s, 3H, CH₃), 6.64 (d, 1H, J=5.5 Hz, Hₐᵣₒₘ), 7.76 (s, 1H, Hₐᵣₒₘ), 8.23 (d, 1H, J=5.5 Hz, Hₐᵣₒₘ); MS (SI) m/z 205 (M+H⁺).

### b) 1-(4-Methoxy-1-m ethyl-1H-pyrrolo[2,3-b]pyridin-3-yl)-3,3-dimethylaminopropenone (13b)

The compound 13b is obtained, according to the procedure described for the preparation of 3a, with a yield of 82% from 12b. M.p.=138-140° C. (MeOH); ¹H NMR (300 MHz, CDCl₃) δ 3.00 (broad s, 6H, CH₃), 3.87 (s, 3H, CH₃), 4.00 (s, 3H, CH₃), 6.08 (d, 1H, J=12.6 Hz, ═CH), 6.63 (d, 1H, J=5.6 Hz, Hₐᵣₒₘ), 7.74 (d, 1H, J=12.6 Hz, ═CH), 7.76 (s, 1H, Hₐᵣₒₘ), 8.23 (d, 1H, J=5.6 Hz, Hₐᵣₒₘ); MS (SI) m/z 260 (M+H⁺).

### c) 3-[(2-amino)pyrimidin-4-yl]-4-methoxy-1-methyl-1H-pyrrolo[2,3-b]pyridine (14b): meriolin 9

The compound 14b is obtained, according to the procedure described for the preparation of 4a, with a yield of 92% from 13b. M.p.>210° C. (MeOH); ¹H NMR (300 MHz, CDCl₃) δ 3.91 (s, 3H, CH₃), 4.02 (s, 3H, CH₃), 4.93 (broad s, 2H, NH₂), 6.65 (d, 1H, J=5.5 Hz, Hₐᵣₒₘ), 7.45 (d, 1H, J=5.3 Hz, Hₐᵣₒₘ), 7.92 (s, 1H, Hₐᵣₒₘ), 8.26 (d, 1H, J=5.3 Hz, Hₐᵣₒₘ), 8.27 (d, 1H, J=5.5 Hz, Hₐᵣₒₘ); MS (SI) m/z 256 (M+H⁺).

The following compounds were prepared in an identical fashion from 3-acetyl-7-azaindoles substituted in the 4 and/or 6 positions:
3-[(2-amino)pyrimidin-4-yl]-4-ethoxy-1-methyl-1H-pyrrolo[2,3-b]pyridine
3-[(2-amino)pyrimidin-4-yl]-4-nitro-1-methyl-1H-pyrrolo[2,3-b]pyridine
3-[(2-amino)pyrimidin-4-yl]-4-fluoro-1-methyl-1H-pyrrolo[2,3-b]pyridine
3-[(2-amino)pyrimidin-4-yl]-1,4-dimethyl-1H-pyrrolo[2,3-b]pyridine
3-[(2-amino)pyrimidin-4-yl]-4-cyano-1-methyl-1H-pyrrolo[2,3-b]pyridine
3-[(2-amino)pyrimidin-4-yl]-4-phenyl-1-methyl-1H-pyrrolo[2,3-b]pyridine
methyl 3-[(2-amino)pyrimidin-4-yl]-1-methyl-1H-pyrrolo[2,3-b]pyridine-4-carboxylate
-4-amino-3-[(2-amino)pyrimidin-4-yl]-1-methyl-1H-pyrrolo[2,3-b]pyridine
3-[(2-amino)pyrimidin-4-yl]-4-propoxy-1-methyl-1H-pyrrolo[2,3-b]pyridine
3-[(2-amino)pyrimidin-4-yl]-6-chloro-1-methyl-1H-pyrrolo[2,3-b]pyridine
3-[(2-amino)pyrimidin-4-yl]-6-bromo-1-methyl-1H-pyrrolo[2,3-b]pyridine
3-[(2-amino)pyrimidin-4-yl]-6-bromo-4-methoxy-1-methyl-1H-pyrrolo[2,3-b]pyridine
3-[(2-amino)pyrimidin-4-yl]-6-cyano-1-methyl-1H-pyrrolo[2,3-b]pyridine

The O-demethylation reaction (HBr/CH₃CO₂H) of the compounds 4b, 5 and 14b gives the derivatives 15-17 with good yields (scheme 5).

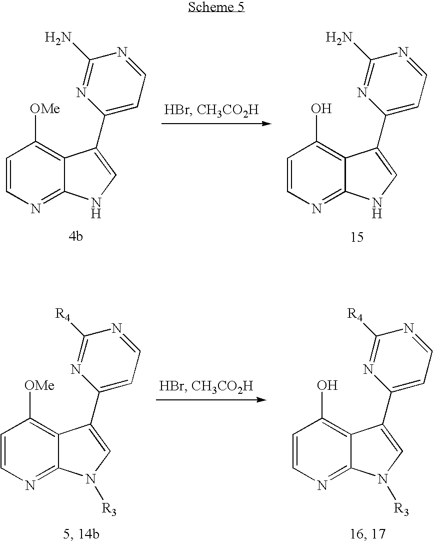

### EXAMPLE 18

### Synthesis of 3-[(2-amino)pyrimidin-4-yl]-4-hydroxy-1H-pyrrolo[2,3-b]pyridine (15): meriolin 2

A solution of 4b (50 mg, 0.20 mmol) in 48% HBr/CH₃CO₂H (6 ml) is heated at reflux for 2 h. After cooling, the solvent is evaporated. The residue obtained is dissolved in AcOEt (10 ml) and then the solution is neutralized by addition of a saturated Na₂CO₃ solution (pH=7-8). The two phases are separated and the aqueous phase collected is washed with AcOEt (2×). The organic phases are combined, dried over MgSO₄ and evaporated. The solid obtained is recrystallized in MeOH to give the compound 15 (43 mg, 90%). M.p.>210° C. (MeOH); ¹H NMR (300 MHz, d₆-DMSO+D₂O) δ 6.51 (d, 1H, J=5.5 Hz, Hₐᵣₒₘ), 7.15 (d, 1H, J=5.5 Hz, Hₐᵣₒₘ), 7.99 (d, 1H, J=5.5 Hz, Hₐᵣₒₘ), 8.17 (d, 1H, 5.5 Hz, Hₐᵣₒₘ), 8.24 (s, 1H, Hₐᵣₒₘ); MS (SI) m/z 228 (M+H⁺).

### EXAMPLE 19

### Synthesis of 3-[(2-amino)pyrimidin-4-yl]-4-hydroxy-1-methyl-1H-pyrrolo[2,3-b]pyridine (16): meriolin 8

The compound 16 is obtained, according to the procedure described for the preparation of 15, with a yield of 92% from 14b. M.p.>210° C. (MeOH); ¹H NMR (300 MHz, d₆-DMSO+D₂O) δ 3.78 (s, 3H, CH₃), 6.56 (d, 1H, J=5.5 Hz, Hₐᵣₒₘ), 7.08 (d, 1H, J=5.5 Hz, Hₐᵣₒₘ), 8.04 (d, 1H, J=5.5 Hz, Hₐᵣₒₘ), 8.16 (d, 1H, J=5.5 Hz, Hₐᵣₒₘ), 8.28 (s, 1H, Hₐᵣₒₘ); MS (SI) m/z 242 (M+H⁺).

### EXAMPLE 20

### Synthesis of 3-(pyrimidin-4-yl)-4-hydroxy-1H-pyrrolo[2,3-b]pyridine (17): meriolin 13

The compound 17 is obtained, according to the procedure described for the preparation of 15, with a yield of 92% from 5. M.p.>210° C. (MeOH); ¹H NMR (300 MHz, d₆-DMSO) δ 6.53 (d, 1H, J=5.1 Hz, Hₐᵣₒₘ), 8.05 (d, 1H, J=5.1 Hz, Hₐᵣₒₘ), 8.12 (d, 1H, J=5.6 Hz, Hₐᵣₒₘ), 8.58 (s, 1H, Hₐᵣₒₘ), 8.71 (d, 1H, J=5.6 Hz, Hₐᵣₒₘ), 9.10 (s, 1H, Hₐᵣₒₘ), 12.42 (broad s, 1H, NH), 14.24 (s, 1H, OH); MS (SI) m/z 213 (M+H⁺).

In the same way, the following compounds were prepared:
3-[(2-amino)pyrimidin-4-yl]-6-bromo-4-hydroxy-1H-pyrrolo[2,3-b]pyridine
3-[(2-amino)pyrimidin-4-yl]-6-bromo-4-hydroxy-1-methyl-1H-pyrrolo[2,3-b]pyridine
3-(pyrimidin-4-yl)-4-hydroxy-1-methyl-1H-pyrrolo[2,3-b]pyridine

### EXAMPLE 21

### Synthesis of 3-(2-aminopyrimidin-4-yl)-4-(2-methoxyethoxyl)-1H-pyrrolo[2,3-b]pyridine: meriolin 7

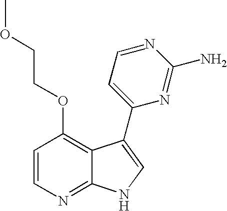

Meriolin 7 was synthesized as described in example 2.

### COMPARATIVE EXAMPLE 1

### Synthesis of 4-methoxy-3-[2-(methylsulfanyl)pyrimidin-4-yl]-1H-pyrrolo[2,3-b]pyridine: meriolin 12.

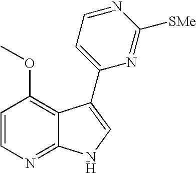

Meriolin 12 was synthesized according to the procedure described in Monatsch. Chem., 2004, 135, 615-627.

### COMPARATIVE EXAMPLE 2

### Synthesis of 3-[(2-amino)pyrimidin-4-yl]-1H-pyrrolo[2,3-b]pyridine: meriolin 1.

Meriolin 1 was synthesized according to the procedure described in Tetrahedron, 2001, 57, 2355-2363.

### COMPARATIVE EXAMPLE 3

### Synthesis of variolin B

Variolin B was synthesized according to the procedure described by Anderson R. J. et al, in 2005 in “Concise Total Syntheses of Variolin B and Deoxyvariolin B”, J. Org. Chem., 2005, 70, 6204-6212.

### II) Tests of the Inhibitory Activity on Protein Kinases of the Meriolins of the Invention

I) Assaying of Protein Kinases

Biochemical Reagents

Sodium orthovanadate, EGTA, EDTA, Mops, β-glycerophosphate, phenyl phosphate, sodium fluoride, dithiothreitol (DTT), glutathione-agarose, glutathione, bovine serum albumin (BSA), nitrophenyl phosphate, leupeptin, aprotinin, pepstatin, trypsin inhibitor from soybean, benzamidine and histone H1 (type III-S) were obtained from Sigma Chemicals. [γ-³³P]-ATP was obtained from Amersham. The GS-1 peptide (YRRAAVPPSPSLSRHSSPHQSpEDEEE) was synthesized by the Peptide Synthesis Unit, Institute of Biomolecular Sciences, University of Southampton, Southampton SO16 7PX, United Kingdom. The CK1-specific peptide substrate (RRKHAAIGSpAYSITA) was kindly donated by Doctors F. Meggio and L. Pinna (Marin et al., 1994).

Buffers

Homogenization buffers: 60 mM β-glycerophosphate, 15 mM p-nitrophenyl phosphate, 25 mM Mops (pH 7.2), 15 mM EGTA, 15 mM MgCl₂, 1 mM DTT, 1 mM sodium vanadate, 1 mM NaF, 1 mM phenyl phosphate, 10 μg leupeptin/ml, 10 μg aprotinin/ml, 10 μg trypsin inhibitor from soybean/ml and 100 μM benzamidine.

Buffer A: 10 mM MgCl₂, 1 mM EGTA, 1 mM DTT, 25 mM Tris-HCl pH 7.5 and 50 μg heparin/ml.

Buffer C: homogenization buffer but 5 mM EGTA, no NaF and no protease inhibitors.

Preparations and Assaying of the Kinases

The kinases were assayed in buffer A or buffer C, at 30° C., at a final ATP concentration of 15 μM. The blank values were subtracted and the activities were calculated as pmol of phosphate incorporated per 10 minutes of incubation. The activities are usually expressed as % (percentage) of the maximum activity, that is to say in the absence of inhibitors. Controls were prepared with appropriate dilutions of dimethyl sulfoxide.

CDK1/cyclin B: was extracted into a homogenization buffer from M-phase starfish oocytes (Marthasterias glacialis) and purified by affinity chromatography on sepharose beads labeled with p9^{CKShs1}, from which it was eluted with free p9^{CKShs1}, as described previously in Meijer et al., (1997) “Biochemical and Cellular Effects of Roscovitine, a Potent and Selective Inhibitor of the Cyclin-Dependent Kinases cdc2, cdk2 and cdk5”, Eur. J. Biochem., 1997, 243, 527-536. The kinase activity was assayed in buffer C, with 1 mg of histone H1/ml, in the presence of 15 μM of [γ-³³P]-ATP (3000 Ci/mmol; 10 mCi/ml), in a final volume of 30 μl. After incubating at 30° C. for 30 minutes, 25 μl aliquots of the supernatant were spotted onto filters made of Whatman P81 phosphocellulose paper and, 20 seconds later, the filters were washed 5 times (for at least 5 (five) minutes each time) in a solution of 10 ml of phosphoric acid/liter of water. The wet filters were subjected to counting in the presence of an ACS scintillation fluid from Amersham.

CDK2/cyclin A (human, recombinant, expressed in insect cells) was assayed as described for CDK1/cyclin B.

CDK5/p25 was reconstituted by mixing equal amounts of recombinant mammalian CDK5 and p25 expressed in E. coli as GST (glutathione S-transferase) fusion protein and purified by affinity chromatography on glutathione-agarose (vectors provided by Doctor L. H. Tsai) (p25 is a truncated version of p35, the 35-kDa CDK5 activator). Its activity was assayed with histone H1 in buffer C as described for CDK1/cyclin B.

CDK7/cyclin H (human, recombinant, expressed in insect cells) was assayed as described for CDK1/cyclin B but using a myelin basic protein (MBP) (1 mg/ml) as substrate.

CDK9/cyclin T (human, recombinant, expressed in insect cells) was assayed as described for CDK1/cyclin B but using a pRB fragment (a.a.773-928) (3.5 μg/assay) as substrate.

GSK-3α/β was purified from porcine brain by affinity chromatography on an immobilized axin (Primot et al., 2003). It was assayed, following a 1/100 dilution in 1 mg BSA/ml and 10 mM DTT, with 5 μl of GS-1 peptide substrate at 4 μM in buffer A, in the presence of 15 μM [γ-³³P]-ATP (3000 Ci/mmol; 10 mCi/ml), in a final volume of 30 μl. After incubating at 30° C. for 30 (thirty) minutes, the 25 μl aliquots of the supernatant were treated as described above.

CK1δ/ε was purified from porcine brain by affinity chromatography on an immobilized axin fragment. It was assayed as described for CDK1 but using a CK1-specific peptide substrate.

DYRK1A kinase originating from the rat, recombinant, expressed in E. coli as GST fusion protein, was purified by affinity chromatography on glutathione-agarose beads and assayed as described for CDK1/cyclin B with the myelin basic protein (1 mg/ml) as substrate.

This study made it possible to demonstrate the particularly marked inhibitory activity of the meriolins with regard to CDKs and in particular CDK9, few inhibitors of which have been described. The meriolins also inhibit GSK-3. These two families of enzymes are particularly implicated in Alzheimer's disease and other neurodegenerative diseases. They also inhibit the kinase DYRK1A, which is directly implicated in Alzheimer's disease and problems related to trisomy 21.

It also emerges from this study that meriolins 3, 4, 5, 6, 15, 16, 17, 18, 22 and 23 show the best inhibitory effect on cell proliferation and that meriolin 19 shows a particularly active and selective effect with regard to the enzyme DYRK1A, which makes it a particularly advantageous candidate for the treatment of neurodegenerative diseases, in particular Alzheimer's disease and trisomy 21.

II) Cell Biology

Antibodies and Chemical Reagents

A Cell Titer 96® kit containing the reagent MTS was purchased from Promega (Madison, Wis., USA). The protease inhibitor cocktail originated from Roche and the fetal calf serum (FCS) originated from Invitrogen. The reagents not listed originated from Sigma, unless otherwise indicated.

Cell Lines and Culturing Conditions

The human neuroblastoma cell line SH-SY5Y was grown in a DMEM medium with L-glutamine originating from Invitrogen (Cergy Pontoise, France), antibiotics and 10% by volume of FCS originating from Invitrogen.

The HEK293 cells were grown in an MEM medium with Glutamax originating from Invitrogen, antibiotics and 10% by volume of FCS.

The general culturing conditions were an atmosphere of 5% of CO₂ and a temperature of 37° C.

The culture plates and other disposable plastic items were supplied by Corning (Corning, N.Y., USA). The drug treatments were carried out on cultures in exponential growth at the time and concentrations indicated. The control experiments were carried out also using appropriate dilutions of DMSO.

Demonstration of the Viability of the Cells

The viability of the cells was determined by measuring the reduction of 3-(4,5-dimethylthiazol-2-yl)-5-(3-carboxymethoxyphenyl)-2-(4-sulfophenyl)-2H/tetrazolium (MTS). The procedure was such as described in detail in Ribas J. et al., 2004, “Cell differentiation, caspase inhibition, and macromolecular synthesis blockage, but not BCL-2 or BCL-XL proteins, protect SH-SYS5 cells from apoptosis triggered by two CDK inhibitory drugs”, Exp. Cell Res., 2004, 195, 9-24.

Demonstration of the Inhibitory Effect of the Meriolins of the Invention with Regard to the Protein Kinases CDK1, CDK2, CDK5, CDK9, GSK3, CM and DYRK1A and with Regard to the SH-SY5Y and HEK293 Cell Lines

Tests were carried out with the meriolins of the invention numbered 2 to 10, 13 to 19 and 22 to 23 and, by way of comparison, with variolin B, meriolin 1 and meriolin 12 described in the prior art.

Thus, variolin B and the meriolins were tested at various concentrations in the assays of seven kinases, as described above.

The IC₅₀ values were calculated from the dose-response curves and are given in the following table 1 in micromoles.

The data for the kinases for the meridianins originate from Gompel et al., Bioorg. Med. Chem. Lett., 2004, 14, 17034707.

These compounds were also tested at various concentrations with regard to their effects on SH-SY5Y and HEK293 cells. The survival of the cells was estimated 48 (forty-eight) hours after the addition of each compound using the MTS reduction assay. The IC₅₀ values were calculated from the dose-response curves and are given in micromoles (mean±standard deviation of two independent measurements carried out in triplicate).

The meriolins of the invention and meriolins 1 and 12 of the prior art tested have the following formula I:

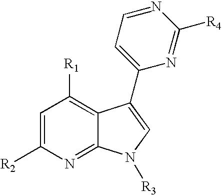

in which the R₁, R₂, R₃ and R₄ substituents are as follows:

| | R₁ | R₂ | R₃ | R₄ |
|---|---|---|---|---|
| Meriolin 1 | H | H | H | NH₂ |
| Meriolin 2 | OH | H | H | NH₂ |
| Meriolin 3 | OCH₃ | H | H | NH₂ |
| Meriolin 4 | OC₂H₅ | H | H | NH₂ |
| Meriolin 5 | OC₃H₇ | H | H | NH₂ |
| Meriolin 6 | OCH(CH₃)₂ | H | H | NH₂ |
| Meriolin 7 | O(CH₂)₂OCH₃ | H | H | NH₂ |
| Meriolin 8 | OH | H | CH₃ | NH₂ |
| Meriolin 9 | OCH₃ | H | CH₃ | NH₂ |
| Meriolin 10 | Cl | H | H | NH₂ |
| Meriolin 12 | OCH₃ | H | H | SCH₃ |
| Meriolin 13 | OH | H | H | H |
| Meriolin 14 | OCH₃ | H | H | H |
| Meriolin 15 | OCH₂C₆H₅ | H | H | NH₂ |
| Meriolin 16 | OCH₂C₆H₁₁ | H | H | NH₂ |
| Meriolin 17 | OC₆H₁₁ | H | H | NH₂ |
| Meriolin 18 | C≡C—(CH₂)₂—CH₃ | H | H | NH₂ |
| Meriolin 19 | (CH₂)₄—CH₃ | H | H | NH₂ |
| Meriolin 22 | (CH₂)₂—C₆H₅ | H | H | NH₂ |
| Meriolin 23 | C₆H₅ | H | H | NH₂ |

The results obtained are given in the following table 1:

TABLE 1

| | CDK1/ | CDK2/ | CDK5/ | CDK9/ | | | |
|---|---|---|---|---|---|---|---|
| Compounds | cyclin B | cyclinA | p25 | cyclin T | GSK-3α/β | CK1 | DYRK1A |
| Variolin B | 0.06 | 0.08 | 0.09 | 0.026 | 0.07 | 0.005 | 008 |
| Meriolin 1 | 0.78 | 0.09 | 0.51 | 0.026 | 0.63 | 0.2 | 0.13 |
| Meriolin 2 | 0.057 | 0.018 | 0.050 | 0.018 | 0.40 | 0.05 | 0.035 |
| Meriolin 3 | 0.17 | 0.011 | 0.17 | 0.006 | 0.23 | 0.2 | 0.029 |
| Meriolin 4 | 0.01 | 0.007 | 0.005 | 0.007 | 0.03 | 0.1 | 0.032 |
| Meriolin 5 | 0.007 | 0.003 | 0.003 | 0.0056 | 0.025 | 0.2 | 0.037 |
| Meriolin 6 | 0.008 | 0.051 | 0.003 | 0.0056 | 0.021 | 0.14 | 0.040 |
| Meriolin 7 | 35.0 | >10 | 14.0 | 5.30 | 63.0 | 100.0 | >10 |
| Meriolin 8 | 1.20 | 1.8 | 5.50 | 1.2 | 4.60 | 2.3 | 1.2 |
| Meriolin 9 | 25.0 | >10 | 73.0 | >10 | >100 | >10 | >10 |
| Meriolin 10 | 0.24 | 0.06 | 0.23 | 0.05 | 2.00 | 3.0 | 0.13 |
| Meriolin 12 | 1.80 | 2.1 | 2.30 | 1.10 | 7.00 | 0.6 | 1.0 |
| Meriolin 13 | 0.9 | 0.7 | 0.7 | 0.25 | 1.8 | 0.9 | 0.9 |
| Meriolin 14 | 1.3 | 0.8 | 1.3 | 0.22 | 1.1 | 0.6 | 0.23 |
| Meriolin 15 | 0.0023 | 0.0016 | 0.002 | 0.0072 | 0.0033 | 0.13 | 0.040 |
| Meriolin 16 | 0.0022 | 0.0012 | 0.003 | — | 0.0060 | >0.1 | 0.045 |
| Meriolin 17 | 0.0029 | 0.0021 | 0.0021 | — | 0.0041 | >0.1 | 0.026 |
| Meriolin 18 | 0.011 | 0.005 | 0.0045 | — | 0.082 | 1.3 | 0.020 |
| Meriolin 19 | 0.350 | 0.195 | 0.20 | — | 1.750 | 8.1 | 0.056 |
| Meriolin 22 | 0.200 | 0.073 | 0.130 | — | 0.800 | — | 0.073 |
| Meriolin 23 | 0.010 | 0.009 | 0.007 | — | 0.080 | — | 0.050 |

It is found, from table 1, that meriolins 3 to 6, 15 to 18 and 22 to 23 exhibit a much greater inhibitory effect on the protein kinases CDK1, CDK2 and CDK9 than variolin B and meriolin 1, which indicates their antiproliferative effect, resulting in cell death.

In the same way, it is seen, from table 1, that meriolins 3 to 6, 15 to 19 and 22 to 23 exhibit an inhibitory effect on the protein kinases CDK5, GSK3, CK1 and DYRK1A which is also much greater than that of variolin B, meriolin 1 and meriolin 12, which indicates a powerful neuroprotective effect.

The apoptotic effect of meriolins 3 to 6, 15 to 18 and 22 to 23 is demonstrated by the results obtained with regard to the survival of SH-SY5Y and HEK293 neuroblastoma cells, which are given in table 2 below.

This exceptionally effective effect with regard to the seven protein kinases tested and with regard to the neuroblastoma cells is particularly marked for meriolin 3, meriolin 4, meriolin 5, meriolin 6, meriolin 15, meriolin 16, meriolin 17, meriolin 18, meriolin 22 and meriolin 23.

Furthermore, it is seen, from table 1, that meriolin 19 exhibits a particularly effective effect with regard to the protein kinase DYRK1A, with a moderate effect with regard to the other protein kinases, which shows not only its effectiveness but also its selectivity with regard to this kinase and renders it particularly appropriate for the treatment of neurodegenerative diseases, such as Alzheimer's disease and trisomy 21.

Demonstration of the Antiproliferative Activity of the Meriolins of the Invention

SH-SY5Y cells were exposed for 24 hours to increasing concentrations of each meriolin and, by way of comparison, of variolin B.

The survival of the cells was estimated by the assaying of the degree of reduction of MTS induced by this exposure.

The results obtained with regard to each meriolin of the invention and with regard to meriolins 1 and 12 and variolin B of the prior art are given in the following table 2.

These results are expressed in the form of the IC₅₀ value, in micromoles.

TABLE 2

| Compound | SH-SY5Y |
|---|---|
| Variolin B | 0.24 |
| Meriolin 1 | 0.67 |
| Meriolin 2 | 0.41 |
| Meriolin 3 | 0.073 |
| Meriolin 4 | 0.081 |
| Meriolin 5 | 0.026 |
| Meriolin 6 | 0.038 |
| Meriolin 7 | >100 |
| Meriolin 8 | >30 |
| Meriolin 9 | >30 |
| Meriolin 10 | 1.92 |
| Meriolin 12 | >100 |
| Meriolin 13 | 28.0 |
| Meriolin 14 | 10.2 |
| Meriolin 15 | 0.013 |
| Meriolin 16 | 0.022 |
| Meriolin 17 | 0.027 |
| Meriolin 18 | 0.100 |
| Meriolin 19 | 13.0 |
| Meriolin 22 | 2.4 |
| Meriolin 23 | 0.088 |

Furthermore, FIG. 1 represents the results obtained for meriolin 3 and meriolin 4. These results are expressed as % of survival with respect to untreated cells.

As is seen from table 2 and FIG. 1, the meriolins of the invention have a much greater antiproliferative effect than that of variolin B.

Demonstration of the Apoptotic Effect of the Meriolins of the Invention

The same experiment as above was carried out but the levels of release of LDH brought about by meriolins 3 and 4 of the invention and variolin B were measured.

The level of release of LDH is representative of the level of mortality of the cells. The higher the level of LDH released, the greater the mortality of the cells.

The results obtained are represented in FIG. 2.

It is seen, from FIG. 2, that the meriolins of the invention bring about cell death.

In Vivo Antitumor Activity

Athymic male nude mice (aged from 5 to 6 weeks) were obtained from the National Cancer Institute. The mice were housed in the animal facility of the Division of Comparative Medicine of Georgetown University. All the studies on the animals were carried out under protocols approved by the Animal Care and Use Committee of Georgetown University. The mice were inoculated by subcutaneous injection into the right posterior flank with 4×10⁶ A4573 cells in 100 μl of Matrigel basic membrane matrix (Becton Dickinson). Xenografts were grown to a mean tumor volume of 129±30 mm³. The compounds tested were first dissolved in either absolute methanol or DMSL (1 volume). A carrier solution was produced using a diluent containing 10% Tween 80, 20% N—N-dimethylacetamide and 70% polyethylene glycol 400 (Fisher Scientific, Pittsburgh, Pa.). The mice were randomly divided into two groups (six animals per group) and the treatment was initiated. One group was treated with meriolin 3, administered by intraperitoneal injection once daily and at a dose of 50 mg/kg for either five days or two series of five days with a pause of two days between each series of five days. The control group received intraperitoneal injections of the carrier solution according to identical programs. All the mice were sacrificed by asphyxia with CO₂. The mice treated with meriolin 3 were euthanized either 7 days after the first injection or after four weeks after the end of the treatment. At these times, the tumors were removed, measured and prepared for TUNEL assays. The primary tumor volumes were calculated by the formula V=(1/2)a×b², where a is the longest tumor axis and b is the shortest tumor axis. The values are given in the form of mean±standard deviation values in quantitative experiments. The statistical analysis of the differences between the groups was carried out by a one-way ANOVA, followed by an unpaired Student's t test.

The results are shown in the appended FIG. 3.

As is seen in FIG. 3, the mean tumor volume, in mm³, only increases slightly over time when the tumor is exposed to meriolin 3, in comparison with exposure to a control, DMSO.

The effects of meriolins 2 and 3 on the survival of various cell lines were also tested at various concentrations in order to determine their effects on eight different cell lines. The survival of the cells was expressed forty eight hours after the addition of each meriolin, using the MTS reduction test. The IC_{S}© values were calculated from dose-response curves and are given in micromoles in table 3 below.

TABLE 3

Survival of the cells
(IC₅₀, μM)
(MTS reduction)

| Cell line | Meriolin 2 | Meriolin 3 |
|---|---|---|
| HCT116 (colon) | 0.080 | 0.94 |
| MDA-MB-231 (breast) | 1.8 | 19.00 |
| PC3 (prostate) | 17.30 | 95.00 |
| Huh7 (hepatoma) | 1.0 | 0.12 |
| F1 (hepatoma) | 2.0 | 0.26 |
| SH-SY5Y (neuroblastoma) | 0.41 | 0.072 |
| HEK293 (embryonic kidney) | 2.6 | 0.38 |
| Human foreskin fibroblasts | 20 | 8.00 |

Thus, it is found that the meriolins of the invention bring about the cell death of cell lines involved in various cancers.

In conclusion, the meriolins of the invention bring about, by apoptosis, the death of cell lines in particular involved in cancer processes. However, this process is not the only one involved in the process bringing about cell death by meriolins, the meriolins also acting as powerful inhibitors of the proliferation of these cells.

These properties of inhibiting the proliferation of cells and of bringing about cell death are certainly very effective in the treatment of tumors but not exclusively.

This is because these properties render the meriolins of the invention appropriate for use in noncancer pathologies, such as renal diseases, including glomerulonephritis, polycystic kidney disease, inflammation, type II diabetes and even neurodegenerative diseases, such as Alzheimer's disease.

Furthermore, although the antiproliferative, apoptotic and antitumor activity of the meriolins of the invention taken alone has been demonstrated in the preceding examples, it will be clearly apparent to a person skilled in the art that the same effects are obtained with combinations of several meriolins according to the invention with one another and also with combinations of at least one meriolin according to the invention with another agent, in particular an antitumor agent, such as, for example, taxol.

In addition, although only the activities of the meriolin compounds of formula I were tested in the preceding examples, it will be clearly apparent to a person skilled in the art that the pharmaceutically acceptable salts of these compounds of formula I will have the same activities and may exhibit additional advantages, such as a better solubility in a physiologically acceptable solvent, reduced side effects, and the like.

The appropriate pharmaceutically acceptable salts are well known in the art. They are in particular the hydrochloride, hydrobromide, sulfate, hydrogensulfate, maleate and fumarate salts of the compounds of formula I.

1.-32. (canceled)

33. A compound of following formula I: 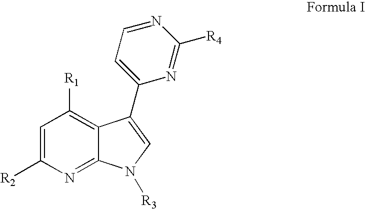
in which:
R₁ is chosen from a halogen or a substituted or unsubstituted C₁-C₁₀ alkyl group, a C₅-C₈ (C₁-C₁₀ alkyl)cycloalkyl group, a C₆-C₁₈ (C₁-C₁₀ alkyl)aryl group, an aromatic or nonaromatic C₅-C₁₂ (C₁-C₁₀ alkyl)heterocyclyl group comprising from one to three heteroatoms, a substituted or unsubstituted alkoxy group, a C₁-C₁₀ fluoroalkoxy group, a C₁-C₁₀ (C₁-C₁₀ alkoxy)alkoxy group, a C₅-C₈ cycloalkoxy group, a C₅-C₈ (C₁-C₁₀ alkoxy)cycloalkyl group, a C₆-C₁₈ (C₁-C₁₀ alkoxy)aryl group, an aromatic or nonaromatic C₅-C₁₂ (C₁-C₁₀ alkoxy)heterocyclyl group comprising from one to three heteroatoms, a substituted or unsubstituted C₂-C₁₀ alkenyl group, a C₅-C₈ (C₂-C₁₀ alkenyl)cycloalkyl group, a C₆-C₁₈ (C₂-C₁₀ alkynyl)aryl group, an aromatic or nonaromatic C₅-C₁₂ (C₂-C₁₀ alkenyl)heterocyclyl group comprising from one to three heteroatoms, a substituted or unsubstituted C₂-C₁₀ alkynyl group, a C₅-C₈ (C₂-C₁₀ alkynyl)cycloalkyl group, a C₆-C₁₈ (C₂-C₁₀ alkynyl)aryl group, an aromatic or nonaromatic C₅-C₁₂ (C₂-C₁₀ alkynyl)heterocyclyl group comprising from one to three heteroatoms, an —OH group, an —OCORₐ group, a —CN group, an —NO₂ group, an —SRₐ group, an —NRₐR_{b} group, an —NHCORₐ group, an —NHSO₂Rₐ group, an —NHSO₂Rₐ group, an —NHCONRₐR_{b} group, an —NHCO₂Rₐ group, a phenyl group, a C₆-C₁₈ aryl group or an aromatic or nonaromatic C₅-C₁₂ heterocyclyl group comprising from one to three heteroatoms,
R₂ represents a hydrogen or halogen atom or, independently of R₁, a group as defined for R₁,
R₃ represents H or an —SO₂Rₐ or —CORₐ, or C₁-C₁₀ alkyl group,
R₄ represents a hydrogen atom or an NH₂ group,
Rₐ and R_{b} represent, each independently of one another, a hydrogen atom or an optionally substituted group chosen from a C₁-C₁₀ alkyl, a C₂-C₁₀ alkenyl, a C₂-C₁₀ alkynyl, a C₅-C₈ cycloalkyl, a C₁-C₁₀ (C₅-C₈ cycloalkyl)alkyl, a C₂-C₁₀ (C₅-C₈ cycloalkyl)alkenyl, a C₂-C₁₀ (C₅-C₈ cycloalkyl)alkynyl, a C₁-C₁₀ (C₅-C₁₂ heterocycle)alkyl, a C₂-C₁₀ (C₅-C₁₂ heterocycloalkyl)alkenyl or a C₂-C₁₀ (C₅-C₁₂ heterocycloalkyl)alkynyl or else Rₐ and R_{b} are bonded together to form, with the nitrogen atom to which they are bonded, an optionally substituted heterocycle chosen from a pyrrolidinyl, piperidinyl, piperazinyl and morpholinyl group,
and the pharmaceutically acceptable salts of this compound of formula I.

34. The compound as claimed in claim 33 having the formula I, in which:
R₁ is chosen from a halogen or a C₁-C₁₀ alkyl, unsubstituted or substituted C₁-C₁₀ alkoxy, C₁-C₁₀ fluoroalkoxy, C₁-C₁₀ (C₁-C₁₀ alkoxy) alkoxy, C₅-C₈ cycloalkoxy, —OH, —OCORₐ, —CN, —NO₂, —SRₐ, —NRₐR_{b}, —NHCORₐ, —NHSO₂Rₐ, —NHCONRₐR_{b}, —NHCO₂Rₐ, phenyl, aryl or heteroaryl group,
R₂ represents a hydrogen atom or, independently of R₁, a halogen or a group as defined for R₁,
R₃ represents H or an —SO₂Rₐ or —CORₐ or alkyl group,
R₄ represents a hydrogen atom or an NH₂ group,
Rₐ and R_{b} represent, each independently of one another, a hydrogen atom or an optionally substituted group chosen from alkyl, alkenyl, alkynyl, cycloalkyl, cycloalkylalkyl, cycloalkylalkenyl, cycloalkylalkynyl, heterocycloalkyl, heterocycloalkylalkyl or heterocycloalkylalkenyl groups or else Rₐ and R_{b} are bonded together to form, with the nitrogen atom to which they are bonded, an optionally substituted heterocycle chosen from a pyrrolidinyl, piperidinyl, piperazinyl or morpholinyl group.

35. The compound as claimed in claim 33 of formula I, in which R₃ is H or CH₃.

36. The compound as claimed in claim 33 having the formula I, in which:
R₁ is chosen from OH, Cl or a methoxy, ethoxy, propoxy, butyloxy, isopropoxy, benzyloxy, cyclohexylmethoxy, cyclohexyloxy, 2-propylethynyl, 2-butylethynyl, 2-cyclohexylethynyl, pheneth-1-ynyl, phenyl, pentyl or phenylethyl group,
R₂ is H or Br,
R₃ is H or CH₃, and
R₄ is H or NH₂.

37. The compound as claimed in claim 33 of formula I, in which:
R₁ is chosen from a methoxy, ethoxy, propoxy, isopropoxy, benzyloxy, cyclohexylmethoxy, cyclohexyloxy, 2-propylethynyl, pentyl, phenyl and phenylethyl group,
R₂ is H or Br,
R₃ is H or CH₃, and
R₄ is H or NH₂.

38. The compound as claimed in claim 33, chosen from:
3-[(2-amino)pyrimidin-4-yl]-4-methoxy-1H-pyrrolo[2,3-b]pyridine of following formula Ia: 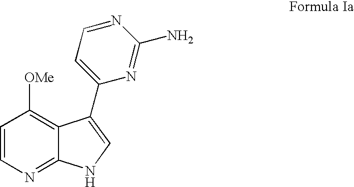
3-[(2-amino)pyrimidin-4-yl]-4-ethoxy-1H-pyrrolo[2,3-b]pyridine of following formula Ib: 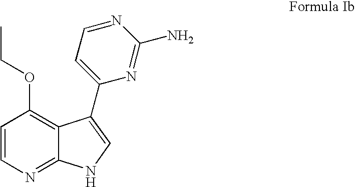
3-[(2-amino)pyrimidin-4-yl]-4-propoxy-1H-pyrrolo[2,3-b]pyridine of following formula Ic: 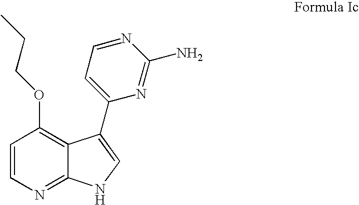
3-[(2-amino)pyrimidin-4-yl]-4-(1-methylethoxy)-1H-pyrrolo[2,3-b]pyridine of following formula Id: 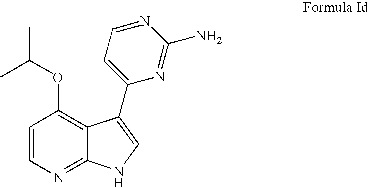
3-[(2-amino)pyrimidin-4-yl]-4-(benzyloxy)-1H-pyrrolo[2,3-b]pyridine of following formula Ie: 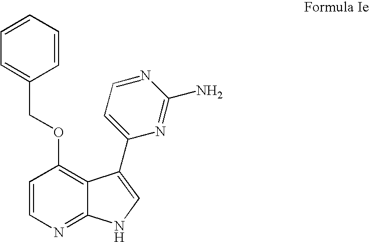
3-[(2-amino)pyrimidin-4-yl]-4-(1-cyclohexylmethoxy)-1H-pyrrolo[2,3-b]pyridine of following formula If: 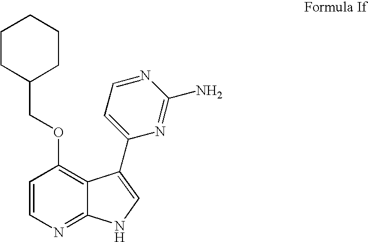
3-[(2-amino)pyrimidin-4-yl]-4-(cyclohexyloxy)-1H-pyrrolo[2,3-b]pyridine of following formula Ig: 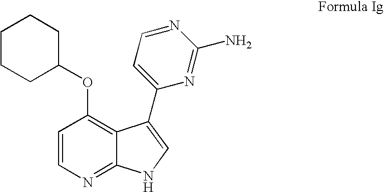
3-[(2-amino)pyrimidin-4-yl]-4-(pent-1-ynyl)-1H-pyrrolo[2,3-b]pyridine of following formula Ih: 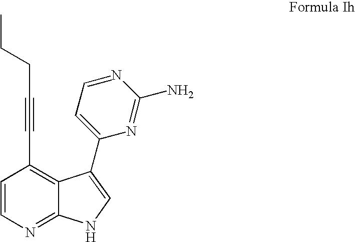
3-[(2-amino)pyrimidin-4-yl]-4-pentyl-1H-pyrrolo[2,3-b]pyridine of following formula Ij: 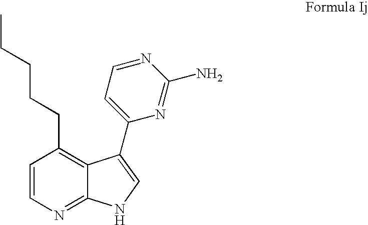
3-[(2-amino)pyrimidin-4-yl]-4-phenethyl-1H-pyrrolo[2,3-b]pyridine of following formula Ik: 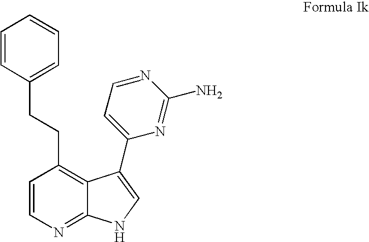
3-[(2-amino)pyrimidin-4-yl]-4-phenyl-1H-pyrrolo[2,3-b]pyridine of following formula Im: 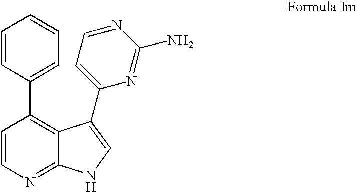
and its pharmaceutically acceptable salts.

39. The compound as claimed in claim 33, chosen from:
3-[(2-amino)pyrimidin-4-yl]-4-(4-methoxybenzyloxy)-1H-pyrrolo[2,3-b]pyridine
3-[(2-amino)pyrimidin-4-yl]-4-(3-methoxybenzyloxy)-1H-pyrrolo[2,3-b]pyridine
3-[(2-amino)pyrimidin-4-yl]-4-(2-methoxybenzyloxy)-1H-pyrrolo[2,3-b]pyridine
3-[(2-amino)pyrimidin-4-yl]-4-(4-chlorobenzyloxy)-1H-pyrrolo[2,3-b]pyridine
3-[(2-amino)pyrimidin-4-yl]-4-(3-chlorobenzyloxy)-1H-pyrrolo[2,3-b]pyridine
3-[(2-amino)pyrimidin-4-yl]-4-(2-chlorobenzyloxy)-1H-pyrrolo[2,3-b]pyridine
3-[(2-amino)pyrimidin-4-yl]-4-(4-fluorobenzyloxy)-1H-pyrrolo[2,3-b]pyridine
3-[(2-amino)pyrimidin-4-yl]-4-(3-fluorobenzyloxy)-1H-pyrrolo[2,3-b]pyridine
3-[(2-amino)pyrimidin-4-yl]-4-(4-hydroxybenzyloxy)-1H-pyrrolo[2,3-b]pyridine
3-[(2-amino)pyrimidin-4-yl]-4-(3-hydroxybenzyloxy)-1H-pyrrolo[2,3-b]pyridine
3-[(2-amino)pyrimidin-4-yl]-4-(pyridin-4-ylmethoxy)-1H-pyrrolo[2,3-b]pyridine
3-[(2-amino)pyrimidin-4-yl]-4-(pyridin-3-ylmethoxy)-1H-pyrrolo[2,3-b]pyridine
3-[(2-amino)pyrimidin-4-yl]-4-(pyridin-2-ylmethoxy)-1H-pyrrolo[2,3-b]pyridine
3-[(2-amino)pyrimidin-4-yl]-4-(pyrimidin-5-ylmethoxy)-1H-pyrrolo[2,3-b]pyridine
3-[(2-amino)pyrimidin-4-yl]-4-(pyridazin-4-ylmethoxy)-1H-pyrrolo[2,3-b]pyridine
3-[(2-amino)pyrimidin-4-yl]-4-(piperidin-4-ylmethoxy)-1H-pyrrolo[2,3-b]pyridine
3-[(2-amino)pyrimidin-4-yl]-4-(1-methanesulfonylpiperidin-4-ylmethoxy)-1H-pyrrolo[2,3-b]pyridine

40. The compound of following formula II: 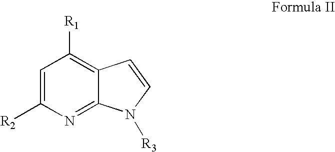
in which
R₁ is an ethoxy, propoxy, butyloxy, isopropoxy, benzyloxy, cyclohexylmethoxy or cyclohexyloxy group,
R₂ is H and
R₃ is H.

41. A process for the synthesis of the compound as claimed in claim 33, comprising:
providing a stage in which at least one compound of formula II is used; and
synthesizing the compound of formula I from the at least one compound of formula II, wherein formula II is 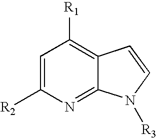
in which
R₁ is an ethoxy, propoxy, butyloxy, isopropoxy, benzyloxy, cyclohexylmethoxy or cyclohexyloxy group,
R₂ is H and
R₃ is H.

42. A medicament comprising the compound of formula I as claimed in claim 33 or one of its pharmaceutically acceptable salts.

43. A pharmaceutical composition, comprising at least one compound of formula I as claimed in claim 33, or at least one of its pharmaceutically acceptable salts, and a pharmaceutically acceptable excipient.

44. A method of manufacture of a medicament for the treatment of disorders and diseases, comprising:
providing at least one compound as claimed in claim 33 or of at least one of its pharmaceutically acceptable salts in the manufacture of a medicament for the treatment of disorders and diseases related to an abnormal proliferation of cells, chosen in particular from a tumor, and kidney diseases, such as glomerulonephritis or polycystic kidney disease.

45. A method of manufacture of a medicament for the treatment of Alzheimer's disease, comprising:
providing at least one compound as claimed in claim 33 or of at least one of its salts in the manufacture of a medicament for the treatment of Alzheimer's disease.

46. A method of manufacture of a medicament for the treatment of trisomy 21, comprising:
providing the compound of formula (Ij) of claim 39 or of at least one of its salts in the manufacture of a medicament for the treatment of trisomy 21.
